(19) United States
(12) Patent Application Publication
(10) Pub. No.: US 2010/0068187 A1
(43) Pub. Date: Mar. 18, 2010

(54) TRANSCRIPTION FACTORS FOR DIFFERENTIATION OF ADULT HUMAN OLFACTORY PROGENITOR CELLS

(76) Inventors: Fred J. Roisen, Prospect, KY (US); Kathleen M. Klueber, Louisville, KY (US); Chengliang Lu, Louisville, KY (US); Xiaodong Zhang, St. Louis, MO (US); Mengsheng Qui, Louisville, KY (US)

Correspondence Address: EVAN LAW GROUP LLC, 600 WEST JACKSON BLVD., SUITE 625, CHICAGO, IL, 60661, US

(21) Appl. No.: 11/909,197

(22) PCT Filed: Aug. 27, 2007
(86) PCT No.: PCT/US2007/076915

§ 371 Date: Dec. 7, 2009

Related U.S. Application Data
(60) Provisional application No. 60/824,217, filed on Aug. 31, 2006.

(51) Int. Cl. A61K 45/00; C12N 5/071; A61P 25/00

(52) U.S. Cl. 424/93.7; 435/366

(57) ABSTRACT

A method of transplantation that includes lineage priming human progenitor cells, to form lineage primed cells and transplanting the lineage primed cells into a patient. The lineage primed cells are selected from the group consisting of oligodendrocytic lineage primed cells, dopaminergic lineage primed cells, and motoneuronal lineage primed cells, and lineage priming has an efficiency of at least 1%.

### FEDERALLY SPONSORED RESEARCH OR DEVELOPMENT

This subject matter of this application may have been funded in part under NIH Grant No. 1920RR15576. The U.S. Government may have rights in this invention.

### BACKGROUND

Stem cells, their differentiated progeny, and the products produced therein, may be capable of unlocking treatments for some of the world's most devastating diseases. Stem cell research has fueled much interest and promise in replacement cell therapy for degenerative diseases. In the context of neurological diseases, replacement cell therapy offers the potential of treating Alzheimer's disease, spinal cord injuries and Parkinson's disease, by replacing aging or damaged tissue with cells displaying physiologically and morphologically compatible properties. However, the benefits and successes of stem cell research are often overshadowed by moral and ethical considerations because the most versatile stem cells used in research and treatments originate from human embryos or aborted fetal tissues. These ethical concerns are often weighed against the ability of stem cells to revolutionize the practice of medicine and improve the quality and length of life.

Besides embryonic or fetal tissue sources, stem cells may be harvested from adult tissues, albeit with some significant limitations. Adult stem cells are often present in only minute quantities, are difficult to isolate and purify, and their number may decrease with age. Furthermore, although stem cells have been isolated from diverse regions of the adult central nervous system (CNS), they can be removed from none of these locations without serious consequences to the donor (A. Gritti, et. al., 1996; V. G. Kukekov, et al., 1997).

The unique regenerative capacity of olfactory neuroepithelium (ONe), found in the nasal cavity, has been well documented in numerous reports. The presence of a progenitor cell population in ONe with the capacity to produce both neurons and their ensheathment and supporting cells is well known (A. L. Calof, et. al., 1998). Still the difficulty has not been in knowing where adult progenitor cells are, but rather in actually locating and isolating adult progenitor cells, and maintaining them in a mitotically active state. Others have established cultures of viable progenitor cells from various sources including embryonic mice (A. L. Calof et. al., 1989, 1998), embryonic rats (A. Kalyani, et. al., 1997; T. Mujaba et. al., 1998; M. S. Rao et. al., 1998; and L. S. Shihabuddin et al., 1997) and neonatal mice and rats (N. K. Mahanthappa et. al., 1993; J. K. McEntire et. al., 2000; S. K. Pixley, 1992, 1994; and M. Satoh and M. Takeuchi, 1995). Cultures from adult mice and rats (A. L. Calof, et. al., 1998, 1989; F. Feron, et. al., 1999; A. Gritti, et. al., 1996; E. D. Laywell, et. al., 1999; N. Liu, et. al., 1998; K. P. A. Mac Donald, et. al., 1996; and J. S. Sosnowski, et. al., 1995), human embryos (A. L. Vescovi et. al., 1999), biopsies from patients with Alzheimer's disease (B. Wolozin et. al., 1993) and normal human adults (F. Feron, et. al., 1999; W. Murrel, et. al., 1996; and B. Wolozin, et. al., 1992) have produced viable ONe cultures, but none have produced progenitor or neurosphere-forming cells. Instead, each of these cultures contained committed neurons, glial and epithelial cells. Only recently have neurosphere-forming cells (which are adult human progenitor cells) been successfully cultured and isolated (F. J. Roisen et al., 2001).

ONe provides a source of viable adult pluripotent progenitor cells, capable of use in research, treatments, drug development, and transplantations, which avoids the ethical concerns associated with use of embryonic and fetal stem cells (F. J. Roisen et al., 2001; and W. Winsted et al, 2005). ONe has a life long regenerative capacity; progenitor cells located within the ONe replace aging and damaged neurons and their sustentacular cells. The accessibility of ONe and proliferative capacity make it a unique source for progenitor cells. Furthermore, the ability to obtain ONe pluripotent progenitor cells from the nasal cavity eliminates the need to use highly invasive and damaging procedures that are currently available to obtain post-embryonic stem cells. In addition, since one of the greatest problems encountered in transplantations is tissue rejection, providing progenitor cells for autologous transplantation eliminates the need to wait for a histocompatible donor and thereby greatly reduces both the frequency and severity of rejection.

Recent progress has been made in developing methods to isolate, culture, and select neurosphere-forming cells (NSFC), which is a regenerative, pluripotent progenitor cell population from the adult ONe. This progenitor cell population remains undifferentiated when maintained in minimal essential medium supplemented with fetal calf serum, but has the potential to differentiate at least along two developmental pathways to yield glial and neuronal lineages. Many NSFC cultures have been established from tissues isolated by endoscopic biopsy from patients ranging in age from 22 years old to 95 years old. Each NSFC culture displays seemingly unlimited self-renewal capacity, being serially passaged in vitro for at least 200 generations. Because each of these cultures are derived from specific individuals, NSFC represents an attractive progenitor cell target for creating differentiated neural cell types for replacement cell therapies that are tailored to unique disease conditions for specific patients. The development of NSFC cultures is described in ADULT HUMAN OLFACTORY PROGENITOR CELLS by Roisen et al., published as WO 03/064601 on 7 Aug. 2003, which is hereby incorporated by reference in its entirety, to the extent not inconsistent with this disclosure.

Neural progenitors obtained from adult human olfactory epithelium following engraftment onto non-human host spinal cord, lead to maintenance of axotomized red nucleus neurons, regeneration of rubrospinal tract neuron axons, and enhanced function (M. Xiao et al. 2005). These remarkable results suggest that the engrafted heterogeneous neural progenitors that encounter a host CNS tissue environment conducive for inducing further development of neural progenitors could respond as replacement cells at the lesion site and provide a permissive environment that facilitates autoregenerative mechanisms in the host. This finding also suggests that the factors governing neural progenitor differentiation may be conserved among vertebrates, particularly among mammals.

Neuronal differentiation depends on inductive signals that include retinoic acid (RA), sonic hedgehog (Shh), and forskolin (FN). Retinoic acid has an important role in fate specification and differentiation of specific neuronal subtypes in the developing CNS, as evidenced by its role in promoting neurite growth of adult mouse dorsal root ganglia neurons and synaptic plasticity in adult mouse hippocampus. Molecular signaling by Shh is critical for the generation of various neuronal cell types including motoneurons and interneurons in the ventral region of the embryonic chicken CNS and the formation of motoneurons and dopaminergic neurons of the embryonic mouse CNS. Finally, FN can stimulate axonal elongation, induce embryonic rat motoneuron survival and potentiate the responsiveness of retinal ganglion cells to trophic factors.

Differentiation is also promoted by proteins that effect transcription of genes necessary for neuronal specification and differentiation. For example, the transcription factor Olig2 has been shown to be essential in the generation of oligodendrocytes and motoneurons in vivo, being expressed in these cells and having a key role in specifying the pan-neuronal properties of developing neurons. Neurotrophic factors, such as glial-derived neurotrophic factor (GDNF) and brain-derived neurotrophic factor (BDNF), also promote neuronal differentiation of embryonic stem cells. Besides playing a role in specification and differentiation of CNS cell types, the expression of many of these differentiation factors continues in the mature terminally differentiated cell type. For example, expression of the Olig2 protein persists in mature oligodendrocytes.

It would be desirable to use human NSFCs as a source to develop specific CNS cell types for replacement cell therapy for patients. Human NSFCs undergo spontaneous differentiation at less than 1% efficiency when NSFCs are cultured in medium containing 10% fetal bovine serum. Human NSFCs also appear refractory to neuronal differentiation when incubated in the presence of neurotrophic factors GDNF or BDNF. Thus, no previous method exists that permits efficient differentiation of human progenitor cells, such as human NSFCs.

### SUMMARY

In a first aspect, the invention is a method of transplantation that includes lineage priming human progenitor cells, to form lineage primed cells and transplanting the lineage primed cells into a patient. The lineage primed cells are selected from the group consisting of oligodendrocytic lineage primed cells, dopaminergic lineage primed cells, and motoneuronal lineage primed cells, and lineage priming has an efficiency of at least 1%.

In a second aspect, the invention is a method for producing lineage primed cells that includes lineage priming human progenitor cells to form lineage primed cells. The lineage priming has an efficiency of at least 1%, and the lineage primed cells are dopaminergic lineage primed cells or motoneuronal lineage primed cells.

In a third aspect, the invention is a lineage priming composition, comprising retinoic acid, and at least one of sonic hedgehog and forskolin.

In a fourth aspect, the invention is a lineage priming composition that includes HB9, and at least one of OIig2 and Ngn2. The composition causes lineage priming of neurosphere forming cell with an efficiency of at least 1%.

In a fifth aspect, the invention is a cell culture that includes neurosphere forming cells and at least 1% dopaminergic lineage primed cells.

In a sixth aspect, the invention is a cell culture that includes neurosphere forming cells and at least 1% motoneuronal lineage primed cells.

### DEFINITIONS

A “pluripotent” cell is one that has an unfixed developmental path, and consequently may differentiate into various differentiated cell types, for example, neurons, oligodendrocytes, astrocytes, ensheathing cells or glial cells. Although pluripotent cells are able to develop into other cell types, various pluripotent cells may be limited in the number of developmental pathways they may travel.

A “progenitor cell” describes any precursor cell, capable of self-renewal, whose daughter cells may commit to differentiate into other cell types (non-progenitor cells). In general, a progenitor cell is capable of extensive proliferation, generating more progenitor cells (self-renewal) as well as more non-progenitor cells. Progenitor cells may divide asymmetrically, with one daughter cell retaining the progenitor cell state and the other being a non-progenitor cell state expressing some other distinct specific function and/or phenotype. Alternatively, some of the progenitor cells in a population may divide symmetrically into progenitor cells, thus maintaining some progenitor cells in the population as a whole, while other cells in the population give rise only to non-progenitor cells. Examples of progenitor cells include embryonic stem cells and cells obtained from olfactory neuroepithelium, bone marrow, fat, and epidermal follicle. Examples of progenitor cells also include any cell derived from a primary cell culture that displays the attributes of progenitor cells. An example of a progenitor cell of this type includes neurosphere forming cells obtained from culturing adult human olfactory neuroepithelium biopsy tissue, as described in ADULT HUMAN OLFACTORY PROGENITOR CELLS by Roisen et al., published as WO 03/064601 on 7 Aug. 2003.

The phrase “human progenitor cell” refers to a regenerative, pluripotent cell from a human that displays the ability to undergo lineage-restricted specification.

A “non-progenitor cell” refers to a cell that derives from a progenitor cell and displays some other distinct specific function and/or phenotype not expressed in the progenitor cell. A non-progenitor cell may differentiate into one or more additional cellular states, as adjudged by the expression of additional functions and phenotypes not expressed in the progenitor cell.

The phrase “lineage-restricted specification” refers to the commitment of a progenitor cell to form a non-progenitor cell type.

The phrase “lineage priming” refers to any action that induces a progenitor cell to undergo lineage-restricted specification.

The phrase “lineage primed cells” refers to cells that originate from a progenitor cell as a result of lineage priming.

The term “adult” refers to a biologically mature animal of the species. For humans, adult refers to a person who is at least sixteen years of age. More preferably, an adult who is human refers to a person having an age of 18 to 100, including 21, 25, 29, 35, 40, 45, 50, 55, 60, 65, 75, 85, and 95 years of age. Most preferably, an adult who is human refers to a person having an age within the range 60 to 85 years of age, including 60, 65, 75, 80, and 85 years of age.

The term “efficiency” refers to the proportion of non-progenitor cells formed from progenitor cells as a result of lineage priming. An example of one method for determining the efficiency of lineage priming is to use immunohistochemistry to visualize a marker associated with only a non-progenitor cell and to count the number of cells having that marker and the number of total cells in the representative population. The efficiency, expressed as a percentage, would be the ratio of cells having the marker to the total cell population, multiplied by one hundred.

A “neurosphere forming cell” (NSFC) is a regenerative, pluripotent progenitor cell population from the adult ONe that displays the ability to form a cluster of about 20 to 80 mitotically active neuronal or glial precursor cells. Morphologically, NSFCs represent a population of neural cells in different stages of maturation formed by a single, clonally expanding progenitor that forms spherical, tightly packed cellular structures. Human NSFCs, as determined by the chromosome structure or reaction with human specific antibodies, have at least two of the following characteristics, and preferably have at least three of the following characteristics, more preferably have at least four of the following characteristics, still more preferably have at least five of the following characteristics and most preferably has all of the following characteristics: (i) divides every 18-24 hours for over 200 passages; (ii) displays immunoreactivity for the marker β-tubulin isotype III that is significantly elevated when the cell is grown on various substratum, such as a matrix coated with a mixture of entacnin, laminin and collagen IV (ECL-matrix) alternatively laminin or fibronectin may also be used; (iii) displays immunoreactivity for β-tubulin isotype III that is much higher than the immunoreactivity of other cell types for this marker; (iv) forms processes on the cell surface upon addition of dibutyryl cAMP to a culture growing on an ECL-matrix; is immunopositive for NCAM marker; or (v) does not require a feeder layer for growth and proliferation. Human NSFCs may not have all of the aforementioned characteristics, but will have at least two of these characteristics simultaneously.

A “cell-restricted lineage pathway” refers to cell states of non-progenitor cells that belong to a common pathway of specification that may lead to a terminally differentiated cell.

An oligodendrocytic lineage primed cell refers to a cell that has at least four of the following characteristics, including characteristic (i); and preferably has at least five of the following characteristics, including characteristic (i); more preferably has at least six of the following characteristics, including characteristic (i); still more preferably has at least seven of the following characteristics, including characteristic (i); and most preferably has all of the following characteristics: (i) expresses amounts of tyrosine hydroxylase (TH) that are either undetectable or below that expressed in dopaminergic neuronal cells; (ii) expresses 2′3′-cyclic nucleotide-3′-phosphohydrolase (CNP); (iii) expresses myelin basic protein (MBP); (iv) expresses galactosylceramide (GalC); (v) expresses RIP; (vi) expresses amounts of glial fibrillary acidic protein (GFAP) that are either undetectable or below that expressed in astrocytes; (vii) expresses amounts of the neuronal marker neuronal nuclear antigen (NeuN) that are either undetectable or below that expressed in neuronal cells; (viii) expresses amounts of the microglial marker OX42 that are either undetectable or below that expressed in microglial cells; or (ix) forms processes that wrap around regions dorsal root ganglia neurons (DRGNs) upon co-culturing the cells with DRGNs. Oligodendrocytic lineage primed cells may not have all of the aforementioned characteristics, but will have at least four of these characteristics simultaneously, including characteristic (i).

A dopaminergic lineage primed cell refers to a cell that has at least four of the following characteristics, including characteristic (i); and preferably has at least five of the following characteristics, including characteristic (i); more preferably has at least six of the following characteristics, including characteristic (i); still more preferably has at least seven of the following characteristics, including characteristic (i); and most preferably has all of the following characteristics: (i) expresses TH; (ii) expresses NF68, (iii) expresses NF160, (iv) expresses NF200, (v) expresses NeuN; (vi) expresses IsI1 and/or IsI2 (IsI1/2); (vii) expresses amounts of vesicular acetylcholine transporter (VAChT) that are either undetectable or below that expressed in TH-negative neuronal cells; (viii) expresses amounts of choline acetyl transferase (ChAT) that are either undetectable or below that expressed in TH-negative neuronal cells; (ix) expresses amounts of GFAP that are either undetectable or below that expressed in astrocytes; (x) expresses amounts of GalC that are either undetectable or below that expressed in oligodendrocyte cells; (xi) expresses amounts of MBP that are either undetectable or below that expressed in oligodendrocyte cells; or (xii) expresses amounts of the microglial marker OX42 that are either undetectable or below that expressed in microglial cells. Dopaminergic lineage primed cells may not have all of the aforementioned characteristics, but will have at least four of these characteristics simultaneously, including characteristic (i).

A motoneuronal lineage primed cell refers to a cell that has at least four of the following characteristics, including characteristic (i); and preferably have at five of the following characteristics, including characteristic (i); more preferably have at least six of the following characteristics, including characteristic (i); still more preferably have at least seven of the following characteristics, including characteristic (i); and most preferably have all of the following characteristics: (i) expresses amounts of TH that are either undetectable or below that expressed in dopaminergic neuronal cells; (ii) expresses NF68, (iii) expresses NF160, (iv) expresses NF200, (v) expresses NeuN; (vi) expresses IsI1/2; (vii) expresses amounts of VAChT that are substantially greater than that expressed in TH-positive neuronal cells; (viii) expresses amounts of ChAT that are substantially greater than that expressed in TH-positive neuronal cells; (ix) expresses amounts of GFAP that are either undetectable or below that expressed in astrocytes; (x) expresses amounts of GalC that are either undetectable or below that expressed in oligodendrocyte cells; (xi) expresses amounts of MBP that are either undetectable or below that expressed in oligodendrocyte cells; (xii) expresses amounts of the microglial marker OX42 that are either undetectable or below that expressed in microglial cells; or (xiii) forms neuromuscular junctions when the cells are co-cultured with skeletal muscle cells, and the location of the junction have synapsin I and ChAT or acetylcholine co-localized. Motoneuronal lineage primed cells may not have all of the aforementioned characteristics, but will have at least four of these characteristics simultaneously, including characteristic (i).

The term “transformation” refers to any alteration in gene expression resulting in a phenotypic change to a cell. Examples of transformation include increasing or decreasing the expression of an endogenous gene by incorporation of lineage priming agents inside cells or by contacting cells with lineage priming agents.

The phrase “transforming the cell” refers to any treatment of a cell with a lineage priming agent that results in transformation of the cell along a cell-restricted lineage pathway.

The phrase “lineage priming agent” includes any composition that is capable of lineage priming. For example, a lineage priming protein is a protein that alone or with other agents is capable of lineage priming. Other examples of lineage priming agents include exogenous gene sequences, including coding sequences, such as open reading frames that encode a protein, and non-coding sequences, such as promoter and enhancer transcriptional elements that can enhance expression of endogenous genes following recombination of such sequences into cells; nucleic acids; and small molecules, such as organic molecules having a mass less than 1,000 daltons, and mixtures thereof.

The term “exogenous” refers to anything that is exposed to a cell or introduced into a cell, which originates from outside the cell.

The term “endogenous” refers to anything that exists in a cell or produced within a cell, and is the antonym of “exogenous.”

The following genes and the products encoded by them, including DNA, RNA, and protein, may come from any vertebrate organism, including human, monkey, mouse, chicken, frog, bovine, horse, sheep, and pig: sonic hedgehog, Olig2, Sox10, NRx2.2, HB9, and Ngn2. The phrase “lineage priming agent” includes each of these genes, variants thereof, and mixtures thereof.

The compound “retinoic acid” refers to retinoic acid and any derivative thereof that can act as a lineage priming agent.

The compound “forskolin” refers to forskolin, such as that obtained from Coleus forskohlii, and any derivative thereof that can act as a lineage priming agent.

The term “vector” refers to any nucleic acid that is capable of expressing an exogenous sequence or recombining with an endogenous sequence following introduction into a cell.

### BRIEF DESCRIPTION OF THE DRAWINGS

FIG. 1 depicts cell neurite analysis, wherein NSFCs were cultured in DFBNM alone (Control) or in medium supplemented as indicated for seven days;

FIGS. 2A-D depict NSFCs that express neuronal and motoneuronal phenotype changes following seven days treatment with RA1 FN5Shh: NSFCs expressed peripherin (FIG. 2A: red), the mature neuronal marker NeuN (FIG. 2B: green), and the motoneuron markers HB9 (FIG. 2C: green) and IsI1/2 (FIG. 2D: green);

FIGS. 2E-F depict NSFCs have increased neuronal restriction and decreased BrdU incorporation compared with controls (FIG. 2A);

FIGS. 3A-D depict increased neurotransmitter and tyrosine hydroxylase expression in NSFCs following a seven day treatment with RA1 FN5Shh: synapsin I (FIG. 3A: red), ChAT (FIG. 3B: green), VAChT (FIG. 3C: red), and tyrosine hydroxylase (FIG. 3D: green);

FIGS. 3E-F depict Western Blot analysis and quantification of protein profiles of NSFCs following a seven day treatment with RA1 FN5Shh;

FIG. 4A depicts immunohistochemical analysis of neuronal and motoneuronal antigen NeuN and IsI1/2 profiles in NSFCs transfected with control vector (C-V), Olig2 and EGFP (O-E), Ngn2 and EGFP (N-E)), HB9 and EGFP (H-E), Olig2 and Ngn2 (O-N), Ngn2 and HB9 (N-H), and Olig2 and HB9 (O-H) for 2 days after 7 days of selection and with or without RFS treatment;

FIG. 4B depicts nestin expression and BrdU incorporation profiles in NSFC transfectants of FIG. 4A;

FIG. 5A depicts a western blot assay of NSFC expression of Olig2, Ngn2, and HB9 after 2 days transfection and seven days selection with RFS treatment;

FIG. 5B depicts quantification of protein profiles presented in FIG. 5A;

FIG. 6A depicts a western blot assay of NSFC expression of neuronal antigens ChAT, VAChT, and TH after 2 days transfection and seven days selection with RFS treatment;

FIG. 6B depicts quantification of protein profiles presented in FIG. 5A;

FIGS. 7A-C depict co-culture studies with NFSCs after two days transfection with Olig2 and HB9 and seven days selection with RFS treatment and illustrate expression of Olig2 (FIG. 7A: red), HB9 (FIG. 7B: red) and motoneuron factor IsI1/2 (FIG. 7C: red); and

FIGS. 7D-I depict co-culture studies with NSFCs after two days transfection with Ngn2 and HB9 (N-H) or Olig2 and HB9 (O-H) and four days selection with RF treatment, NFSCs were seeded onto purified chicken skeletal muscle for three days with RS treatment, wherein the NSFCs expressed GFP (A-G, I: red) and the neurites were found in contact with muscle straps, where they formed presumptive neuromuscular junctions that expressed ChAT (FIG. 7D: red), Ach (FIG. 7E: red), Ach in muscle strap (FIG. 7F: red), and synapsin I (FIGS. 7H-I: red).

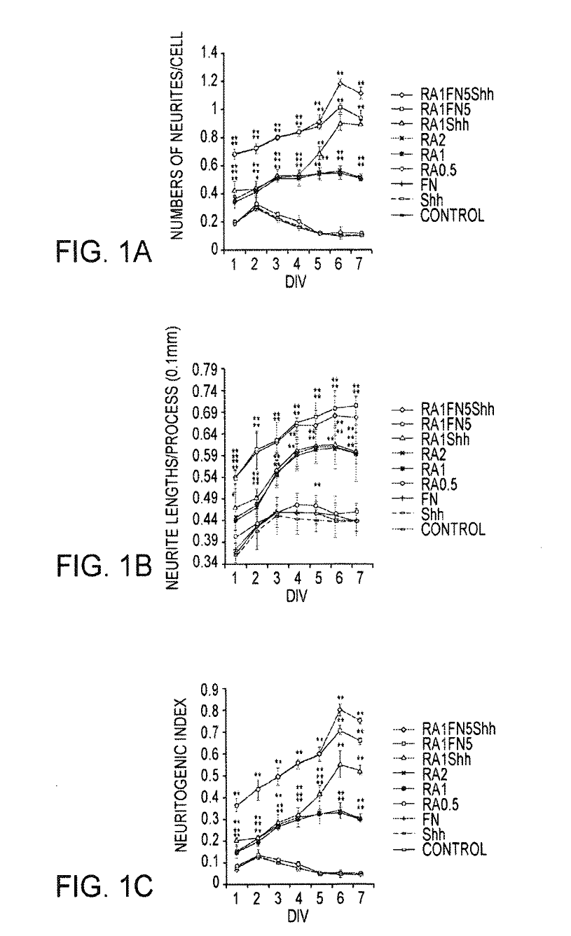

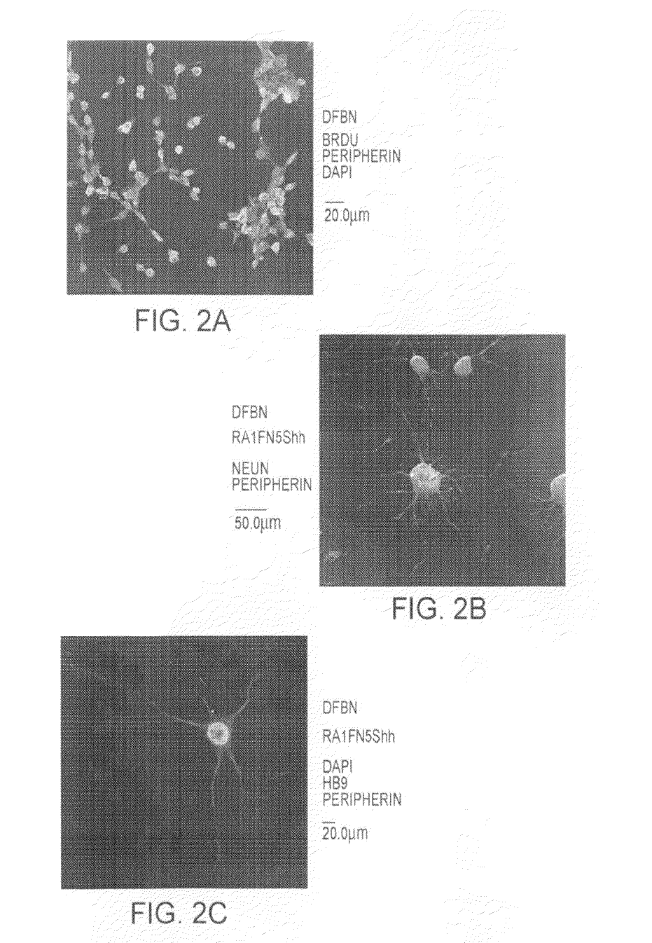

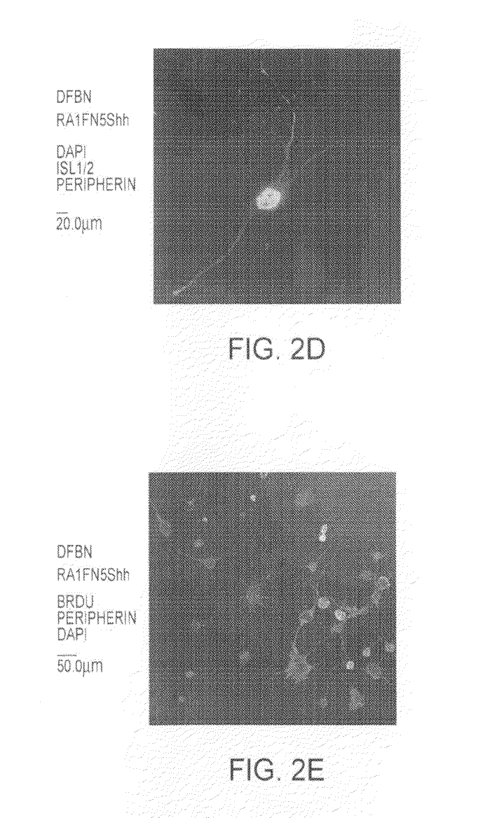

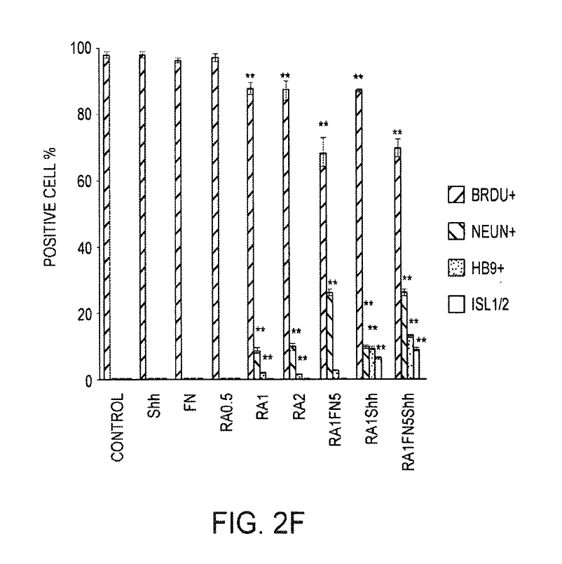

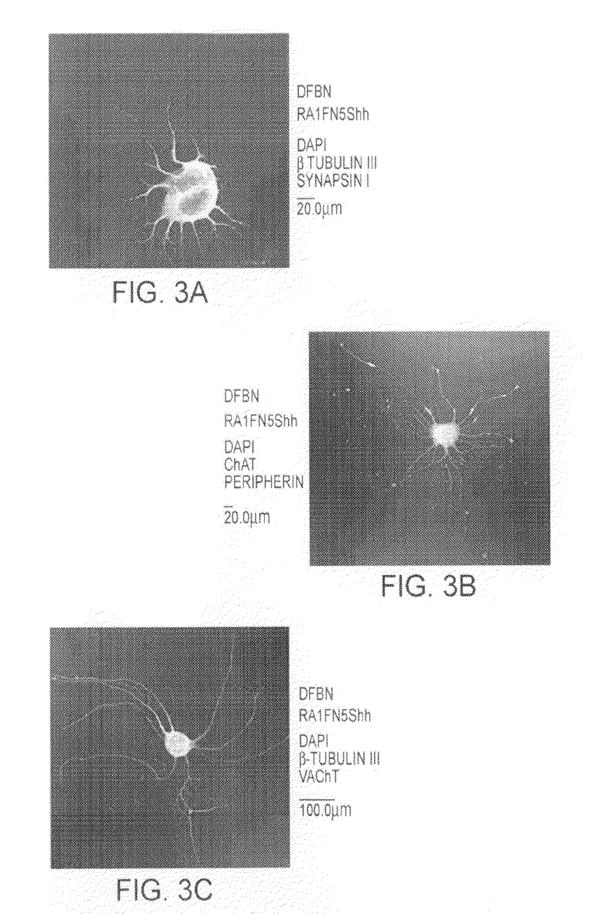

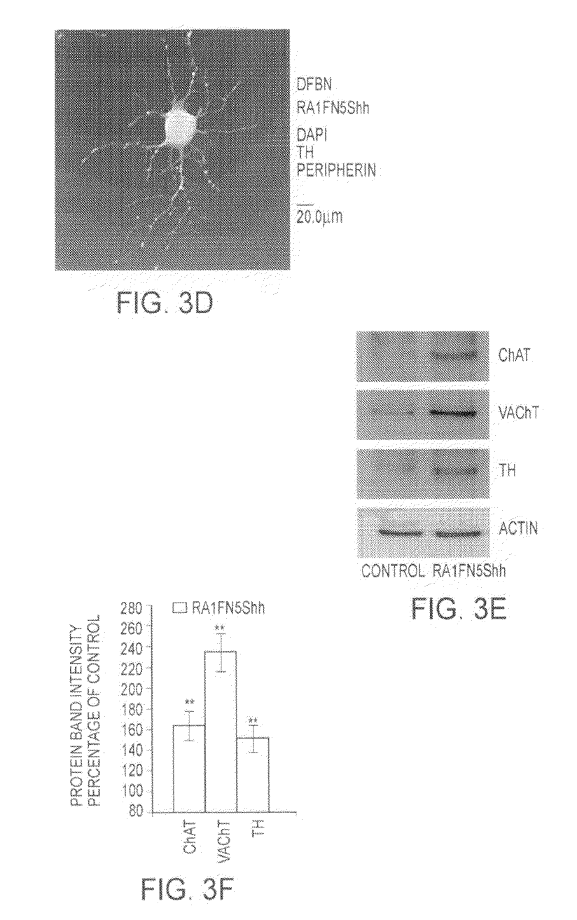

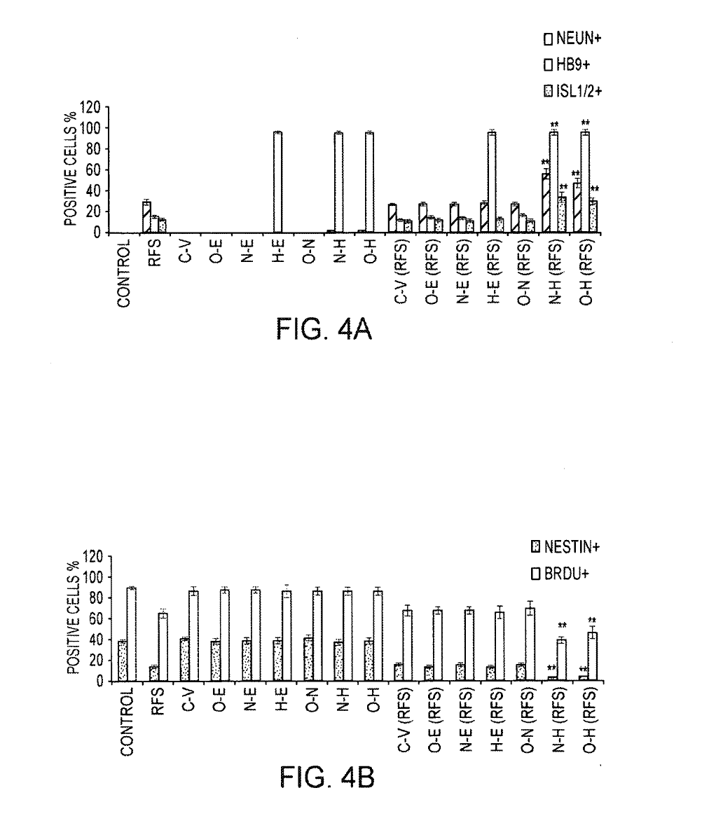

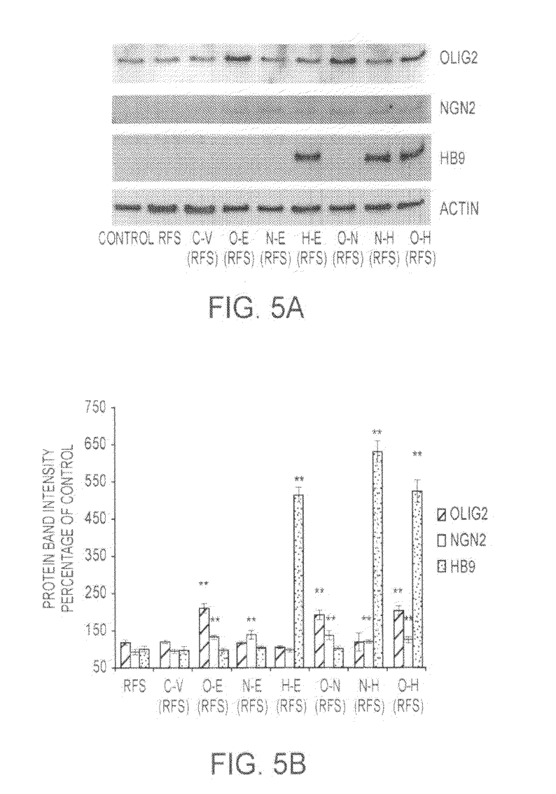

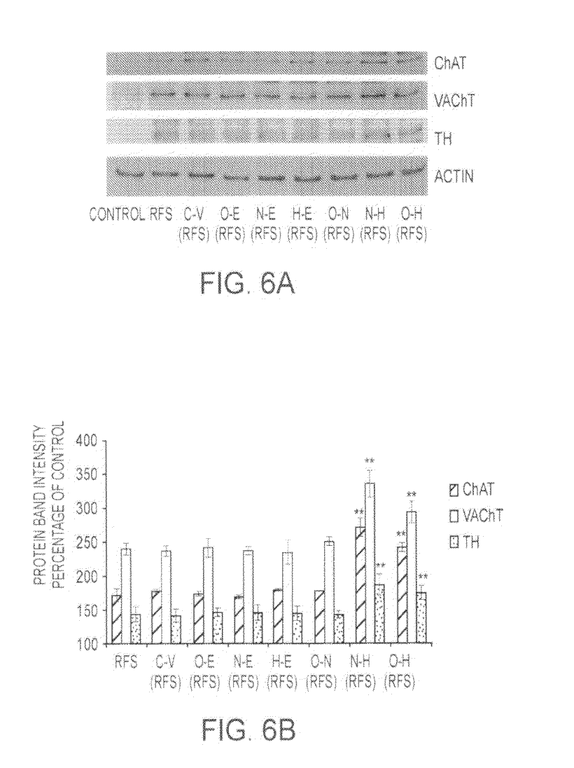

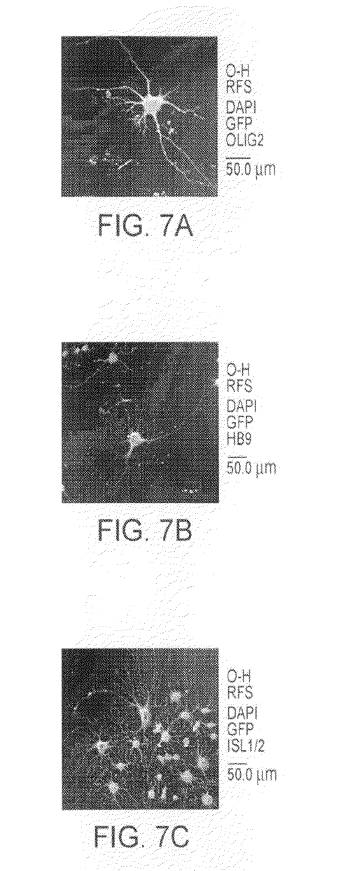

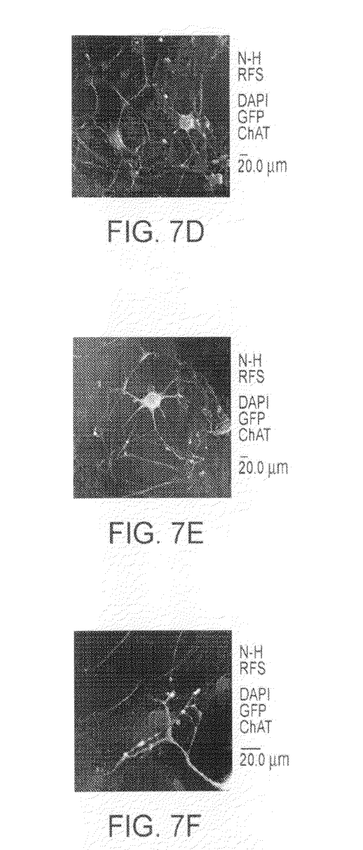

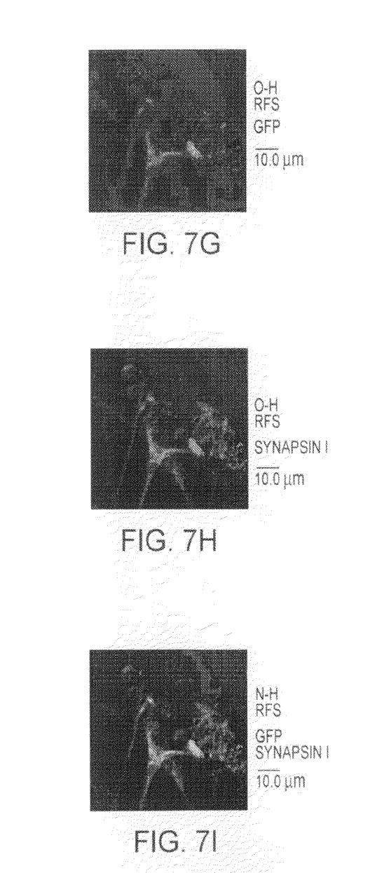

### DETAILED DESCRIPTION

The present invention makes use of the discovery of methods for treating human progenitor cells, such as neurosphere-forming cells (NSFCs), to produce lineage primed cells suitable for use in cell replacement strategies, such as autologous transplantation, disease modeling, and diagnostic testing methodologies. Lineage priming of NSFCs results in cell-restricted lineage pathways leading to oligodendrocytic lineage primed cells, dopaminergic lineage primed cells, or motoneuronal lineage primed cells. An important aspect of the present invention is the use of lineage priming agents to cause an efficiency of lineage priming greater than that previously achieved with conventional culturing conditions. These lineage primed cell populations have therapeutic utility for transplantation into patients with central nervous system trauma or neurodegenerative diseases, and are particularly useful for autologous transplantation.

The human progenitor cells from adult olfactory neuroepithelium remain relatively undifferentiated when maintained in a minimal medium, such as MEM10, or when exposed to a variety of defined media and trophic factors. These NSFC cultures appear to have an immature neuronal default, in which more than 97% of the cells express both β tubulin III and peripherin and about one-half the population of the cells expresses nestin. This suggests that the NSFC cultures obtained from adult human olfactory neuroepithelium may be different from embryonic and/or other types of neural stem cells. However, the human NSFC cultures have characteristics of neural progenitor cells. For example, the cells do not appear to express the astrocytic marker GFAP, microglial marker OX42, oligodendrocyte markers GalC or MBP, or mature neuronal markers NeuN, HB9, IsI1/2, VAChT, ChAT and TH, each of which is indicative of a central nervous system cell type that has undergone lineage-restricted specification beyond the progenitor stage.

To determine whether human NSFC cultures can undergo lineage priming along cell-restricted lineage pathways using lineage priming agents, NSFC cultures were treated simultaneously with different concentrations and combinations of RA, FN, and Shh in vitro. Depending upon the treatment regimen, the NSFC cultures undergo lineage priming to produce neurites having characteristics of motoneuronal lineage primed cells and dopaminergic lineage primed cells. NSFCs do not form these lineage primed cell types upon treatment with RA, FN, or Shh alone.

Lineage priming may be affected by treating NSFC cultures for seven days with RA in the presence of FN, Shh, or a combination of FN and Shh. Preferably, at least 1 μM RA in combination with FN at a concentration of 1 μM to 10 μM, including 2 μM, 3 μM, 5 μM, 7 μM and 8 μM, resulted in motoneuronal and dopaminergic lineage primed cells. Alternatively, at least 1 μM RA in combination with Shh at a concentration of 5 nM to 20 nM, including 10 nM, 13 nM, 15 nM, and 17 nM, results in motoneuronal and dopaminergic lineage primed cells. More preferably, at least 1 μM RA in combination with FN at a concentration of 1 μM to 10 μM, including 2 μM, 3 μM, 5 μM, 7 μM and 8 μmM, and Shh at a concentration of 5 nM to 20 nM, including 10 nM, 13 nM, 15 nM, and 17 nM results in motoneuronal and dopaminergic lineage primed cells. Most preferably, at least 1 μM RA in combination with 5 μM FN and 15 nM Shh results in motoneuronal and dopaminergic lineage primed cells. None of these treatments affect cell viability.

Following treatment of NSFC cultures with 1 μM RA in combination with 5 μM FN and 15 nM Shh for seven days, the resultant motoneuronal and dopaminergic lineage primed cells express mature neuronal antigens. Approximately 97% of the NSFCs were β tubulin III+ and peripherin+, about 82% were Tau+, and about 86% were α-internexin+. Furthermore about 31% of the treated NSFCs expressed NF68 localized in the cell soma while about 27% of the cells expressed NF160 and 24% of the cells expressed NF200 in the soma and neuritic processes. By contrast, nestin expression decreased in the treated cells to about 17% and no GFAP, OX42, GalC, nor MBP was detected. Moreover, labeling experiments confirmed that NSFCs treated with 1 μM RA in combination with 5 μM FN and/or 15 nM Shh did not incorporate significant amounts of BrdU, but did induce expression of NeuN. Thus, the NSFCs undergo lineage-restricted specification along a neuronal pathway that includes motoneuronal and dopaminergic lineage primed cells.

The treated NSFCs display morphological and functional characteristics of motoneuronal lineage primed cells. NSFCs treated with 1 μM RA in combination with 5 μM FN and 15 nM Shh form motoneuronal lineage primed cells, as evidenced by their ability to form functional connections with muscle fibers. Furthermore, confocal imaging revealed co-localization at neuromuscular junctions of synapsin I and acetylcholine (ACh) or choline acetyltransferase (ChAT), which packages acetylcholine into vesicles for release at neuromuscular junctions.

The resultant motoneuronal lineage primed cells also display numerous spines at their terminals. Electron microscopic examination demonstrated the presence of vesicles within these spines that were shown by immunocytochemistry to contain synapsin I and vesicular acetylcholine transporter (VAChT), the functional transporter for the neurotransmitters ACh and ChAT. Western blot analysis of the cells following the seven day treatment with 1 μM RA in combination with 5 μM FN and 15 nM Shh confirmed the presence of these neurotransmitters.

In addition to these phenotypic and morphologic changes, lineage priming of NSFCs with RA in combination with Shh or with Shh and FN display increased expression of transcription factors found in motoneuronal lineage primed cells. In particular, lineage priming of NSFCs with either 1 μM RA in combination with 15 nM Shh, or 1 μM RA in combination with 5 μM FN and 15 nM Shh, resulted in induction of the motoneuron transcription factors homeo box HB9 (HB9) and Islet 1 and/or 2 (IsI1/2). However, lineage priming of NSFCs does not result with 0.5 μM RA, 5 μM FN or 15 nM Shh alone, as evidenced by the failure of the treated-NSFCs to form motoneuronal lineage primed cells and dopaminergic lineage primed cells.

Furthermore, about 12% of the treated NSFCs form dopaminergic lineage primed cells, as evidenced by the expression of the dopaminergic neuronal specific antigen, tyrosine hydroxylase. The TH-positive cells failed to express ChAT or VAChT, thereby delineating these cells as dopaminergic lineage primed cells rather than motoneuronal lineage primed cells.

Without wanting to be limited to any particular theory, these results suggest that lineage priming by RA, in combination with FN and/or Shh, may cause transformation events leading to the lineage-restricted specification of NSFCs along particular cell-restricted lineage pathways for specific neuronal cell types. For example, the transcription factor Olig2 has been shown to be essential in the generation of motoneurons, being expressed in motoneuron progenitors and having a key role in specifying the pan-neuronal properties of developing neurons. Olig2 also directs expression of motoneuron transcription factors, including Islet1/2 (IsI1/2), the LIM-homeodomain gene, and the MNR/HB9 homeobox gene in neural progenitor cells. The transcription factor Neurogenin 2 (Ngn2) is required for the generation of mouse motoneurons, the development of mouse cranial sensory ganglia, and functions as a neuronal lineage factor. Since both IsI1/2 and HB9 are defined markers for motoneurons and their progenitors, treatment of NSFCs with RA/FN/Shh may induce the expression of these transcription factors to affect lineage priming along cell-restricted lineage pathways.

To assess whether the introduction of lineage priming agents into NSFCs could effect lineage priming along neuronal cell-restricted lineage pathways, NSFCs were transfected with vectors that simultaneously cause expression of Ngn2 and HB9 and incubated in the presence of 1 μM RA in combination with 5 μM FN for four days and in the presence of 1 μM RA in combination with 15 nM Shh for three additional days (“RFS treatment”). The morphology of the NSFCs transfected with vectors that cause expression of both Ngn2 and HB9 and combined with RFS treatment for seven days underwent lineage-restricted specification that resulted in motoneuronal lineage primed cells. Greater than 97% of the transfected NSFCs expressed Ngn2, HB9, β tubulin III, and peripherin; demonstrated increased expression of the neuronal markers NF68, NF160, NF200, NeuN, and ChAT, and the neuronal transcription factor IsI1/2. By contrast, the expression of nestin and incorporation of BrdU was decreased, and no markers for other cell lineages were detected in the transfected cells, including GalC, MBP, GFAP, or OX42. The neurite lengths and numbers of NSFCs were also increased following transfection with Ngn2 and HB9, selection, and RFS treatment as compared to cells exposed to the RFS treatment without transfection.

Transfection of NSFCs with vectors that simultaneously cause expression of Olig2 and HB9 and combined with RFS treatment induced lineage-restricted specification associated with formation of motoneuronal lineage primed cells and dopaminergic lineage primed cells. For example, lineage priming of NSFCs by these agents resulted in expression of the neuronal markers NeuN, VAChT, ChAT, TH, and the motoneuronal marker IsI1/2, as well as increased neurite lengths and numbers. By contrast, the expression of nestin and incorporation of BrdU decreased. Thus, Olig2 can mimic the effects of Ngn2, in combination with HB9, for lineage priming of NSFCs to form motoneuronal lineage primed cells and dopaminergic lineage primed cells.

Lineage priming of NSFCs to form motoneuronal lineage primed cells and dopaminergic lineage primed cells following introduction and expression of OIig2 and HB9 or Ngn2 and HB9 is not observed following RFS treatment alone, nor with transfection with control vectors, single genes, Olig2 and Ngn2 combined with RFS treatment, Olig2 and EGFP, Ngn2 and EGFP, HB9 and EGFP, nor OIig2 and Ngn2 alone. Thus, lineage priming of NSFCs to form motoneuronal lineage primed cells and dopaminergic lineage primed cells can be driven by OIig2 and HB9 or Ngn2 and HB9 when supplemented by RFS treatment.

In addition to its role in specifying motoneuronal and dopaminergic cell differentiation, the transcription factor Olig2 participates in specifying oligodendrocyte differentiation. During development, oligodendrocytes arise from restricted loci of neuroepithelial precursor cells in the ventral neural tube under the influence of Shh. In the early stages of oligodendrogenesis, the transcription factors Olig1 and Olig2 are initially expressed in oligodendrocyte-generative zones of the neuroepithelium. As oligodendrocyte progenitors leave the ventricular zone, Olig1/2 expression is retained in oligodendrocyte progenitors and persists in immature oligodendrocytes. Molecular and genetic studies have demonstrated that expression of Olig1/2 is required for oligodendrocyte lineage determination in vivo. Oligodendrocyte progenitors acquire expression of two additional transcription factors, Sox10 and Nkx2.2, before or after the progenitors migrate into the white matter. Both Sox10 and Nkx2.2 appear to regulate oligodendrocyte differentiation.

To assess whether the introduction of Olig2, Sox10, and/or Nkx2.2 into NSFCs could affect lineage priming of these progenitors to form oligodendrocytic lineage primed cells, NSFCs were transfected with vectors that simultaneously cause expression of Olig2 and NRx2.2, or Sox10 and NRx2.2. When the NSFCs were transfected with Olig2 and NRx2.2 simultaneously, lineage-restricted specification occurred with the formation of oligodendrocytic lineage primed cells. The cells expressed both Olig2 and NRx2.2 proteins, as confirmed by immunohistochemistry. The cells did not express the early oligodendrocyte precursor marker O4, but instead expressed more mature oligodendrocytic markers, including 2′3′-cyclic nucleotide-3′-phosphohydrolase (CNP). Furthermore, the cells coexpressed GalC and CNP, as well as RIP and myelin basic protein (MBP). No mature markers for other cell lineages were detected, including GFAP (astrocyte marker), NeuN (neuronal marker) or OX42 (microglial marker). Oligodendrocytic lineage primed cells were also obtained upon lineage priming of NSFCs by transfection with vectors that cause simultaneous expression of Sox10 and NRx2.2.

Lineage priming of NSFCs to form oligodendrocytic lineage primed cells was not observed for NSFCs cultured in defined medium (DFB27) or incubated with RA, NF, or Shh, or combinations thereof, or NSFCs transfected with vectors that cause expression of Olig2, NRx2.2, or Sox10 alone or with control vectors. Thus, the presence of the transcription factors Olig2 and NRx2.2, or Sox10 and NRx2.2, is sufficient to direct lineage-restricted specification of human NSFCs along a cell-restricted lineage pathway toward oligodendrocytic lineage primed cells.

To investigate whether the oligodendrocytic lineage primed cells formed direct axonal associations, control NSFCs and lineage primed cells were maintained on top of an established dorsal root ganglia (DRG) neuronal layer for 10 to 14 days. No direct axonal association was detected when control NSFCs were incubated with the DRG neuronal layer. By contrast, lineage primed cells expressing Olig2 and NRx2.2, or Sox10 and NRx2.2, and co-cultured with DRG neurons formed multiple processes that often were observed in direct contact with the DRG neurons. As demonstrated by confocal microscopy, the lineage primed NSFC processes were observed wrapped around individual regions of DRG neurons.

Lineage priming of NSFCs with conventional culture media conditions, such as 10% fetal bovine serum, occurs with an efficiency of less than 1%. By contrast, the efficiency of lineage priming of NSFCs with the lineage priming agents described in the present invention is at least 1%. Preferably, the efficiency of lineage priming of NSFCs is at least 5%. More preferably, the efficiency of lineage priming is at least 10%. Most preferably, the efficiency of lineage priming falls within the range from 5% to 95%, including 10%, 20%, 30%, 40%, 50%, 60%, 75%, and 90%. The efficiency of lineage priming can be determined in a number of ways, including using immunocytochemical analysis to determine the proportion of lineage primed cells of a particular type formed in an NSFC population treated with a lineage priming agent.

Lineage priming of NSFCs may result in mixed cell populations containing both NSFCs and one or more different types of lineage primed cells. For example, NSFCs, motoneuronal lineage primed cells, and dopaminergic lineage primed cells are present following treatment of NSFC cultures with 1 μM RA in combination with 5 μM FN and 15 nM Shh for seven days. Each of these cell populations display specific markers on the cell surfaces that distinguish lineage primed cell types from one another and from NSFCs, and this characteristic may be used in a method to select homogeneous cell populations of a particular type. One such method is the use of an antibody that recognizes as an antigen a specific lineage primed cell marker that is not expressed on other lineage primed cells of a different type or on NSFCs. The antibody may be immobilized onto a solid matrix, such as a resin or bead, preferably a magnetic bead, and used to bind antigen-containing cells of a particular type (for example, NFSCs or a specific lineage primed cell populations). Lineage primed cells that lack the antigen are separated from the antigen-containing lineage primed cells by recovering the solid matrix containing the antibody-bound cells and washing away the unbound cells. Dopaminergic linear primed cells, for example, may be selected from a mixed population containing NSFCs as well as motoneuronal and dopaminergic lineage primed cells using an antibody specific for an antigen expressed specifically by dopaminergic lineage primed cells (for example, TH). These techniques may be adapted to select NSFCs from the original cultures containing olfactory neuroepithelium and to recover NSFCs following a lineage priming treatment. Examples of such selection techniques are described by Othman et al. 2005(a) and Othman et al. 2005(b).

These results demonstrate that lineage priming of NSFCs induced lineage-restricted specification along neuronal cell-restricted lineage pathways to produce several non-progenitor CNS cell types, including oligodendrocytic lineage primed cells, dopaminergic lineage primed cells, or motoneuronal lineage primed cells. Combinations of lineage priming agents may be used for lineage priming of the NSFC cultures and include the following robust regimens: (1) incubation of NSFCs in medium containing RA combined with FN and/or Shh; (2) directed expression of Olig2 and HB9, or Ngn2 and HB9, in NSFC transfectants, particularly when supplemented by incubation of the transfectants in the presence of RA, FN, and Shh; and (3) directed expression of Olig2 and NRx2.2, or Sox10 and NRx2.2, in NSFC transfectants. These regimens have utility in establishing CNS cell types having a morphologic and lineage-restricted phenotype. Such cellular materials are useful for replacement cellular therapy strategies for patients suffering from degenerative CNS diseases.

The foregoing description of methods for lineage priming human NSFCs along neuronal cell-restricted lineage pathways to produce several non-progenitor CNS cell types, including oligodendrocytic lineage primed cells, dopaminergic lineage primed cells, or motoneuronal lineage primed cells is applicable to other sources of human progenitor cells. Thus, human progenitor cells obtained from other sources, such as embryonic stem cells, sources of adult human progenitor cells other than the olfactory neuroepithelium, or reconstructed adult human progenitor cells derived from human somatic cells, are suitable for generating oligodendrocytic lineage primed cells, dopaminergic lineage primed cells, or motoneuronal lineage primed cells.

Polynucleotides

One aspect of the invention pertains to isolated nucleic acid molecules that encode Olig2, HB9, Ngn2, Sox10, or NRx2.2, or biologically-active portions thereof. Also included in the invention are nucleic acid fragments sufficient for use as hybridization probes to identify Olig2, HB9, Ngn2, Sox10, or NRx2.2-encoding nucleic acids (for example, Olig2, HB9, Ngn2, Sox10, or NRx2.2 mRNAs) and fragments for use as polymerase chain reaction (PCR) primers for the amplification and/or mutation of OIig2, HB9, Ngn2, Sox10, or NRx2.2 molecules. A “nucleic acid molecule” includes DNA molecules (for example, cDNA or genomic DNA), RNA molecules (for example, mRNA), analogs of the DNA or RNA generated using nucleotide analogs, and derivatives, fragments and homologs. The nucleic acid molecule may be single-stranded or double-stranded, but preferably comprises double-stranded DNA.

1. Probes

Probes are nucleic acid sequences of variable length, preferably between at least about 10 nucleotides (nt), 100 nt, or many (for example, 6,000 nt) depending on the specific use. Probes are used to detect identical, similar, or complementary nucleic acid sequences. Longer length probes can be obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies. Probes are substantially purified oligonucleotides that will hybridize under stringent conditions to at least optimally 12, 25, 50, 100, 150, 200, 250, 300, 350 or 400-consecutive sense strand nucleotide sequence of OIig2, HB9, Ngn2, Sox10, or NRx2.2, or an anti-sense strand nucleotide sequence of these sequences; or of a naturally occurring mutant of these sequences.

The full- or partial length native sequence for example may be used to identify and isolate similar (homologous) sequences (Ausubel et al., 1987; Sambrook, 1989), such as: (1) full-length or fragments of Olig2, HB9, Ngn2, Sox10, or NRx2.2 cDNA from a cDNA library from any species (for example, human, murine, feline, canine, fish, bird, and frog), (2) from cells or tissues, (3) variants within a species, and (4) homologues and variants from other species. To find related sequences that may encode related genes, the probe may be designed to encode unique sequences or degenerate sequences. Sequences may also be genomic sequences including promoters, enhancer elements and introns of native sequence for Olig2, HB9, Ngn2, Sox10, or NRx2.2.

For example, an Olig2, HB9, Ngn2, Sox10, or NRx2.2 coding region in another species may be isolated using such probes. A probe of about 40 bases is designed, based on an Olig2, HB9, Ngn2, Sox10, or NRx2.2, and made. To detect hybridizations, probes are labeled using, for example, radionuclides such as ³²P or ³⁵S, or enzymatic labels such as alkaline phosphatase coupled to the probe via avidin-biotin systems. Labeled probes are used to detect nucleic acids having a complementary sequence to that of an Olig2, HB9, Ngn2, Sox10, or NRx2.2 sequences in libraries of cDNA, genomic DNA or mRNA of a desired species.

Such probes can be used as a part of a diagnostic test kit for identifying cells or tissues which mis-express an Olig2, HB9, Ngn2, Sox10, or NRx2.2, such as by measuring a level of an Olig2, HB9, Ngn2, Sox10, or NRx2.2 in a sample of cells from a subject for example, detecting Olig2, HB9, Ngn2, Sox10, or NRx2.2 mRNA levels or determining whether a genomic Olig2, HB9, Ngn2, Sox10, or NRx2.2 has been mutated or deleted.

2. Isolated Nucleic Acid

An isolated nucleic acid molecule is separated from other nucleic acid molecules that are present in the natural source of the nucleic acid. Preferably, an isolated nucleic acid is free of sequences that naturally flank the nucleic acid (that is, sequences located at the 5′- and 3′-termini of the nucleic acid) in the genomic DNA of the organism from which the nucleic acid is derived. For example, in various embodiments, isolated Olig2, HB9, Ngn2, Sox10, or NRx2.2 molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (for example, brain, heart, liver, spleen, etc.). Moreover, an isolated nucleic acid molecule, such as a cDNA molecule, can be substantially free of other cellular material or culture medium when produced by recombinant techniques, or of chemical precursors or other chemicals when chemically synthesized.

PCR amplification techniques can be used to amplify Olig2, HB9, Ngn2, Sox10, and NRx2.2 using cDNA, mRNA or alternatively, genomic DNA, as a template and appropriate oligonucleotide primers. Such nucleic acids can be cloned into an appropriate vector and characterized by DNA sequence analysis. Furthermore, oligonucleotides corresponding to Olig2, HB9, Ngn2, Sox10, or NRx2.2 sequences can be prepared by standard synthetic techniques, for example, an automated DNA synthesizer.

3. Oligonucleotide

An oligonucleotide comprises a series of linked nucleotide residues, which oligonucleotide has a sufficient number of nucleotide bases to be used in a PCR reaction or other application. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of Olig2, HB9, Ngn2, Sox10, or NRx2.2, or a complement thereof. Oligonucleotides may be chemically synthesized and may also be used as probes.

4. Complementary Nucleic Acid Sequences; Binding

In another embodiment, an isolated nucleic acid molecule for use with the invention comprises a nucleic acid molecule that is a complement of the nucleotide sequence of Olig2, HB9, Ngn2, Sox10, or NRx2.2 or a portion of such nucleotide sequence (for example, a fragment that can be used as a probe or primer or a fragment encoding a biologically-active portion of an Olig2, HB9, Ngn2, Sox10, or NRx2.2). A nucleic acid molecule that is complementary to the nucleotide sequence of the human Olig2, HB9, Ngn2, Sox10, or NRx2.2 is one that is sufficiently complementary to the nucleotide sequence that it can hydrogen bond with little or no mismatches to the desired nucleotide sequence, thereby forming a stable duplex.

“Complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Nucleic acid fragments are at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full-length sequence. Fragments may be derived from any contiguous portion of a nucleic acid or amino acid sequence of choice.

5. Derivatives, and Analogs

Derivatives are nucleic acid sequences or amino acid sequences formed from the native compounds either directly or by modification or partial substitution. Analogs are nucleic acid sequences or amino acid sequences that have a structure similar to, but not identical to, the native compound but differ from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include molecules comprising regions that are substantially homologous to the nucleic acids or proteins of the invention, in various embodiments, by at least about 80%, 90%, or 95% identity (with a preferred identity of 80-98%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions (Ausubel et al., 1987).

6. Homology

A “homologous nucleic acid sequence” or “homologous amino acid sequence,” or variations thereof, refer to sequences characterized by homology at the nucleotide level or amino acid level as discussed above. Homologous nucleotide sequences encode those sequences coding for isoforms of Olig2, HB9, Ngn2, Sox10, or NRx2.2. Isoforms can be expressed in different tissues of the same organism as a result of, for example, alternative splicing of RNA. Alternatively, different genes can encode isoforms. Homologous nucleotide sequences include nucleotide sequences encoding for an Olig2, HB9, Ngn2, Sox10, or NRx2.2 of species other than humans, including vertebrates, and thus can include, for example, frog, mouse, rat, rabbit, dog, cat, cow, horse, fish, bird, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human Olig2, HB9, Ngn2, Sox10, or NRx2.2. Homologous nucleic acid sequences include those nucleic acid sequences that encode conservative amino acid substitutions (see below) within Olig2, HB9, Ngn2, Sox10, or NRx2.2, as well as a polypeptide possessing Olig2, HB9, Ngn2, Sox10, or NRx2.2 biological activity as a lineage priming agent.

7. Open Reading Frames

The open reading frame (ORF) of an Olig2, HB9, Ngn2, Sox10, or NRx2.2 gene encodes an Olig2, HB9, Ngn2, Sox10, or NRx2.2. An ORF is a nucleotide sequence that has a start codon (ATG) and terminates with one of the three “stop” codons (TAA, TAG, or TGA). In this invention, however, an ORF may be any part of a coding sequence that may or may not comprise a start codon and a stop codon. To achieve a unique sequence, preferable Olig2, HB9, Ngn2, Sox10, or NRx2.2 ORFs encode at least 50 amino acids.

Olig2, HB9, Ngn2, Sox10, and NRx2.2 Polypeptides

1. Mature

An Olig2, HB9, Ngn2, Sox10, or NRx2.2 can encode a mature Olig2, HB9, Ngn2, Sox10, or NRx2.2. A “mature” form of a polypeptide or protein disclosed in the present invention is the product of a naturally occurring polypeptide or precursor form or proprotein. The naturally occurring polypeptide, precursor or proprotein includes, for example, the full-length gene product, encoded by the corresponding gene. Alternatively, it may be defined as the polypeptide, precursor or proprotein encoded by an open reading frame described herein. The product “mature” form arises, for example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from post-translational modification other than a proteolytic cleavage event. Such additional processes include, for example, glycosylation, myristoylation or phosphorylation. In general, a mature polypeptide or protein may result from the operation of only one of these processes, or a combination of any of them.

2. Active

An active Olig2, HB9, Ngn2, Sox10, or NRx2.2 polypeptide or Olig2, HB9, Ngn2, Sox10, or NRx2.2 polypeptide fragment retains a biological and/or an immunological activity similar, but not necessarily identical, to a lineage priming activity of a naturally-occurring (wild-type) Olig2, HB9, Ngn2, Sox10, or NRx2.2 polypeptide of the invention, including mature forms. A particular biological assay, such as lineage priming, with or without dose dependency, can be used to determine Olig2, HB9, Ngn2, Sox10, or NRx2.2 activity. A nucleic acid fragment encoding a biologically-active portion of Olig2, HB9, Ngn2, Sox10, or NRx2.2 can be prepared by isolating a portion of the corresponding gene encodes a polypeptide having an Olig2, HB9, Ngn2, Sox10, or NRx2.2 biological activity expressing the encoded portion of Olig2, HB9, Ngn2, Sox10, or NRx2.2 (for example, by recombinant expression in vitro) and assessing the lineage priming activity of the encoded portion of Olig2, HB9, Ngn2, Sox10, or NRx2.2. Immunological activity refers to the ability to induce the production of an antibody against an antigenic epitope possessed by a native Olig2, HB9, Ngn2, Sox10, or NRx2.2; biological activity refers to a function, either inhibitory or stimulatory, caused by a native Olig2, HB9, Ngn2, Sox10, or NRx2.2 that excludes immunological activity.

Olig2, HB9, Ngn2, Sox10, or NRx2.2 Nucleic Acid Variants and Hybridization

1. Variant Polynucleotides, Genes and Recombinant Genes.

The invention further encompasses nucleic acid molecules that differ from the nucleotide sequences encoded by the Olig2, HB9, Ngn2, Sox10, or NRx2.2 due to degeneracy of the genetic code and thus encode the same Olig2, HB9, Ngn2, Sox10, or NRx2.2.

In addition to the endogenous Olig2, HB9, Ngn2, Sox10, or NRx2.2 sequences, DNA sequence polymorphisms that change the amino acid sequences of the Olig2, HB9, Ngn2, Sox10, or NRx2.2 may exist within a population. For example, allelic variation among individuals will exhibit genetic polymorphism in an Olig2, HB9, Ngn2, Sox10, or NRx2.2. The terms “gene” and “recombinant gene” refer to nucleic acid molecules comprising an open reading frame (ORF) encoding an Olig2, HB9, Ngn2, Sox10, or NRx2.2, preferably a vertebrate Olig2, HB9, Ngn2, Sox10, or NRx2.2. Such natural allelic variations can typically result in 1-5% variance in an Olig2, HB9, Ngn2, Sox10, or NRx2.2. Any and all such nucleotide variations and resulting amino acid polymorphisms in an Olig2, HB9, Ngn2, Sox10, or NRx2.2, which are the result of natural allelic variation and that do not alter the functional activity of an Olig2, HB9, Ngn2, Sox10, or NRx2.2 are within the scope of the invention.

Moreover, Olig2, HB9, Ngn2, Sox10, or NRx2.2 from other species that have a nucleotide sequence that differs from the human sequence are contemplated. Nucleic acid molecules corresponding to natural allelic variants and homologues of an Olig2, HB9, Ngn2, Sox10, or NRx2.2 cDNAs of the invention can be isolated based on their homology to an Olig2, HB9, Ngn2, Sox10, or NRx2.2 using cDNA-derived probes to hybridize to homologous Olig2, HB9, Ngn2, Sox10, or NRx2.2 sequences under stringent conditions.

“Olig2, HB9, Ngn2, Sox10, or NRx2.2 variant polynucleotide” or “Olig2, HB9, Ngn2, Sox10, or NRx2.2 variant nucleic acid sequence” means a nucleic acid molecule which encodes an active Olig2, HB9, Ngn2, Sox10, or NRx2.2 that (1) has at least about 80% nucleic acid sequence identity with a nucleotide acid sequence encoding a full-length native Olig2, HB9, Ngn2, Sox10, or NRx2.2, (2) a full-length native Olig2, HB9, Ngn2, Sox10, or NRx2.2 lacking the signal peptide, (3) an extracellular domain of an Olig2, HB9, Ngn2, Sox10, or NRx2.2, with or without the signal peptide, or (4) any other fragment of a full-length Olig2, HB9, Ngn2, Sox10, or NRx2.2. Ordinarily, an Olig2, HB9, Ngn2, Sox10, or NRx2.2 variant polynucleotide will have at least about 80% nucleic acid sequence identity, more preferably at least about 81%, 82%, 83%, 84%, 85%, 86%, 87%, 88%, 89%, 90%, 91%, 92%, 93%, 94%, 95%, 96%, 97%, 98%, nucleic acid sequence identity and yet more preferably at least about 99% nucleic acid sequence identity with the nucleic acid sequence encoding a full-length native Olig2, HB9, Ngn2, Sox10, or NRx2.2 variant polynucleotide may encode a full-length native Olig2, HB9, Ngn2, Sox10, or NRx2.2 lacking the signal peptide, an extracellular domain of an Olig2, HB9, Ngn2, Sox10, or NRx2.2, with or without the signal sequence, or any other fragment of a full-length Olig2, HB9, Ngn2, Sox10, or NRx2.2. Variants do not encompass the native nucleotide sequences.

Ordinarily, Olig2, HB9, Ngn2, Sox10, or NRx2.2 variant polynucleotides are at least about 30 nucleotides in length, often at least about 60, 90, 120, 150, 180, 210, 240, 270, 300, 450, 600 nucleotides in length, more often at least about 900 nucleotides in length, or more.

“Percent (%) nucleic acid sequence identity” with respect to Olig2, HB9, Ngn2, Sox10, or NRx2.2-encoding nucleic acid sequences identified herein is defined as the percentage of nucleotides in a candidate sequence that are identical with the nucleotides in the Olig2, HB9, Ngn2, Sox10, or NRx2.2 sequence of interest, after aligning the sequences and introducing gaps, if necessary, to achieve the maximum percent sequence identity. Alignment for purposes of determining % nucleic acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

When nucleotide sequences are aligned, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) can be calculated as follows: % nucleic acid sequence identity=(W/Z)×100

where

W is the number of nucleotides cored as identical matches by the sequence alignment program's or algorithm's alignment of C and D

and

Z is the total number of nucleotides in D.

When the length of nucleic acid sequence C is not equal to the length of nucleic acid sequence D, the % nucleic acid sequence identity of C to D will not equal the % nucleic acid sequence identity of D to C.

2. Stringency

Homologs (that is, nucleic acids encoding an Olig2, HB9, Ngn2, Sox10, or NRx2.2 derived from species other than human) or other related sequences (example, paralogs) can be obtained by low, moderate or high stringency hybridization with all or a portion of the particular human sequence as a probe using methods well known in the art for nucleic acid hybridization and cloning.

The specificity of single stranded DNA to hybridize complementary fragments is determined by the “stringency” of the reaction conditions. Hybridization stringency increases as the propensity to form DNA duplexes decreases. In nucleic acid hybridization reactions, the stringency can be chosen to either favor specific hybridizations (high stringency), which can be used to identify, for example, full-length clones from a library. Less-specific hybridizations (low stringency) can be used to identify related, but not exact, DNA molecules (homologous, but not identical) or segments.

DNA duplexes are stabilized by: (1) the number of complementary base pairs, (2) the type of base pairs, (3) salt concentration (ionic strength) of the reaction mixture, (4) the temperature of the reaction, and (5) the presence of certain organic solvents, such as formamide which decreases DNA duplex stability. In general, the longer the probe, the higher the temperature required for proper annealing. A common approach is to vary the temperature: higher relative temperatures result in more stringent reaction conditions. Ausubel et al. (1987) provides an excellent explanation of stringency of hybridization reactions.

To hybridize under “stringent conditions” describes hybridization protocols in which nucleotide sequences at least 60% homologous to each other remain hybridized. Generally, stringent conditions are selected to be about 5° C. lower than the thermal melting point (Tₘ) for the specific sequence at a defined ionic strength and pH. The Tₘ is the temperature (under defined ionic strength, pH and nucleic acid concentration) at which 50% of the probes complementary to the target sequence hybridize to the target sequence at equilibrium. Since the target sequences are generally present at excess, at Tₘ, 50% of the probes are occupied at equilibrium.

(a) High Stringency

“Stringent hybridization conditions” conditions enable a probe, primer or oligonucleotide to hybridize only to its target sequence. Stringent conditions are sequence-dependent and will differ. Stringent conditions comprise: (1) low ionic strength and high temperature washes (for example, 15 mM sodium chloride, 1.5 mM sodium citrate, 0.1% sodium dodecyl sulfate at 50° C.); (2) a denaturing agent during hybridization (for example, 50% (v/v) formamide, 0.1% bovine serum albumin, 0.1% Ficoll, 0.1% polyvinylpyrrolidone, 50 mM sodium phosphate buffer (pH 6.5; 750 mM sodium chloride, 75 mM sodium citrate at 42° C.); or (3) 50% formamide. Washes typically also comprise 5×SSC (0.75 M 10 NaCl, 75 mM sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. Preferably, the conditions are such that sequences at least about 65%, 70%, 75%, 85%, 90%, 95%, 98%, or 99% homologous to each other typically remain hybridized to each other. These conditions are presented as examples and are not meant to be limiting.

(b) Moderate Stringency

“Moderately stringent conditions” use washing solutions and hybridization conditions that are less stringent (Sambrook, 1989), such that a polynucleotide will hybridize to the entire, fragments, derivatives or analogs of Olig2, HB9, Ngn2, Sox10, or NRx2.2. One example comprises hybridization in 6×SSC, 5×Denhardt's solution, 0.5% SDS and 100 mg/ml denatured salmon sperm DNA at 55° C., followed by one or more washes in 1×SSC, 0.1% SDS at 37° C. The temperature, ionic strength, etc., can be adjusted to accommodate experimental factors such as probe length. Other moderate stringency conditions are described in (Ausubel et al., 1987; Kriegler, 1990).

(c) Low Stringency

“Low stringent conditions” use washing solutions and hybridization conditions that are less stringent than those for moderate stringency (Sambrook, 1989), such that a polynucleotide will hybridize to the entire, fragments, derivatives or analogs of Olig2, HB9, Ngn2, Sox10, or NRx2.2. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency, such as those for cross-species hybridizations are described in (Ausubel et al., 1987; Kriegler, 1990; Shilo and Weinberg, 1981).

3. Conservative Mutations

In addition to naturally-occurring allelic variants of Olig2, HB9, Ngn2, Sox10, or NRx2.2, changes can be introduced by mutation into the corresponding sequences that incur alterations in the amino acid sequences of the encoded Olig2, HB9, Ngn2, Sox10, or NRx2.2 that do not alter the Olig2, HB9, Ngn2, Sox10, or NRx2.2 function. For example, nucleotide substitutions leading to amino acid substitutions at “non-essential” amino acid residues can be made in the corresponding gene sequences. A “non-essential” amino acid residue is a residue that can be altered from the wild-type sequences of the Olig2, HB9, Ngn2, Sox10, or NRx2.2 without altering their biological activity as a lineage priming agent, whereas an “essential” amino acid residue is required for such biological activity. For example, amino acid residues that are conserved among the Olig2, HB9, Ngn2, Sox10, or NRx2.2 genes between different species of the invention are predicted to be particularly non-amenable to alteration. Amino acids for which conservative substitutions can be made are well-known in the art.

Useful conservative substitutions are shown in Table A, “Preferred substitutions.” Conservative substitutions whereby an amino acid of one class is replaced with another amino acid of the same type fall within the scope of the subject invention so long as the substitution does not materially alter the biological activity of the compound as a lineage priming agent. If such substitutions result in a change in biological activity, then more substantial changes are introduced and the products screened for an Olig2, HB9, Ngn2, Sox10, or NRx2.2 polypeptide's biological activity as a lineage priming agent.

TABLE A
Preferred Substitutions

| Original | | Preferred |
|---|---|---|
| residue | Exemplary substitutions | Substitutions |
| Ala (A) | Val, Leu, Ile Val | Val |
| Arg (R) | Lys, Gln, Asn Lys | Lys |
| Asn (N) | Gln, His, Lys, Arg Gln | Gln |
| Asp (D) | Glu | Glu |
| Cys (C) | Ser | Ser |
| Gln (Q) | Asn | Asn |
| Glu (E) | Asp | Asp |
| Gly (G) | Pro, Ala | Ala |
| His (H) | Asn, Gln, Lys, Arg | Arg |
| Ile (I) | Leu, Val, Met, Ala, Phe, Leu Norleucine | Leu |
| Leu (L) | Norleucine, Ile, Val, Met, Ala, Ile Phe | Ile |
| Lys (K) | Arg, Gln, Asn | Arg |
| Met (M) | Leu, Phe, Ile | Leu |
| Phe (F) | Leu, Val, Ile, Ala, Tyr | Leu |
| Pro (P) | Ala | Ala |
| Ser (S) | Thr | Thr |
| Thr (T) | Ser | Ser |
| Trp (W) | Phe Tyr | Tyr |
| Tyr (Y) | Trp, Phe, Thr, Ser | Phe |
| Val (V) | Ile, Leu, Met, Phe, Ala, Leu Norleucine | Leu |

Non-conservative substitutions that effect (1) the structure of the polypeptide backbone, such as a β-sheet or α-helical conformation, (2) the charge or (3) hydrophobicity, or (4) the bulk of the side chain of the target site can modify an Olig2, HB9, Ngn2, Sox10, or NRx2.2 polypeptide's function or immunological identity. Residues are divided into groups based on common side-chain properties as denoted in Table B. Non-conservative substitutions entail exchanging a member of one of these classes for another class. Substitutions may be introduced into conservative substitution sites or more preferably into non-conserved sites.

TABLE B
Amino Acid Classes

| Classes | Amino acids |
|---|---|
| hydrophobic | Norleucine, Met, Ala, Val, Leu, Ile |
| neutral/hydrophilic | Cys, Ser, Thr |
| acidic | Asp, Glu |
| basic | Asn, Gln, His, Lys, Arg |
| disrupt chain conformation | Gly, Pro |
| aromatic | Trp, Tyr, Phe |

The variant polypeptides can be made using methods known in the art such as oligonucleotide-mediated (site-directed) mutagenesis, alanine scanning, and PCR mutagenesis. Site-directed mutagenesis (Carter, 1986; Zoller and Smith, 1987), cassette mutagenesis, restriction selection mutagenesis (Wells et al., 1985) or other known techniques can be performed on the cloned DNA to produce the Olig2, HB9 and Ngn2 variant DNA (Ausubel et al., 1987; Sambrook, 1989).

In one embodiment, the isolated nucleic acid molecule comprises a nucleotide sequence encoding a protein, wherein the protein comprises an amino acid sequence at least about 45%, preferably 60%, more preferably 70%, 80%, 90%, and most preferably about 95% homologous to human Olig2, HB9, Ngn2, Sox10, or NRx2.2.

Olig2, HB9, Ngn2, Sox10, or NRx2.2 Polypeptides

One aspect of the invention pertains to isolated Olig2, HB9, Ngn2, Sox10, or NRx2.2, and biologically-active portions derivatives, fragments, analogs or homologs thereof. Also provided are polypeptide fragments suitable for use as immunogens to raise anti-Olig2, HB9, Ngn2, Sox10, or NRx2.2 antibodies. In one embodiment, a native Olig2, HB9, Ngn2, Sox10, or NRx2.2 can be isolated from cells or tissue sources by an appropriate purification scheme using standard protein purification techniques. In another embodiment, Olig2, HB9, Ngn2, Sox10, or NRx2.2 are produced by recombinant DNA techniques. Alternative to recombinant expression, an Olig2, HB9, Ngn2, Sox10, or NRx2.2 can be synthesized chemically using standard peptide synthesis techniques.

1. Polypeptides

An Olig2, HB9, Ngn2, Sox10, or NRx2.2 polypeptide includes the amino acid sequence of an Olig2, HB9, Ngn2, Sox10, or NRx2.2. The invention also includes a mutant or variant protein any of whose residues may be changed from the corresponding residues found for each gene, while still encoding a protein that maintains its Olig2, HB9, Ngn2, Sox10, or NRx2.2 biological activities and physiological functions as a lineage priming agent, or a functional fragment thereof.

2. Variant Olig2, HB9, Ngn2, Sox10, or NRx2.2 Polypeptides

In general, an Olig2, HB9, Ngn2, Sox10, or NRx2.2 variant that preserves an Olig2, HB9, Ngn2, Sox10, or NRx2.2-like function as a lineage priming agent and includes any variant in which residues at a particular position in the sequence have been substituted by other amino acids, and further includes the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

“Olig2, HB9, Ngn2, Sox10, or NRx2.2 polypeptide variant” means an active Olig2, HB9, Ngn2, Sox10, or NRx2.2 polypeptide having at least: (1) about 80% amino acid sequence identity with a full-length native sequence Olig2, HB9, Ngn2, Sox10, or NRx2.2 polypeptide sequence, (2) an Olig2, HB9, Ngn2, Sox10, or NRx2.2 polypeptide sequence lacking the signal peptide, (3) an extracellular domain of an Olig2, HB9, Ngn2, Sox10, or NRx2.2 polypeptide, with or without the signal peptide, or (4) any other fragment of a full-length Olig2, HB9, Ngn2, Sox10, or NRx2.2 polypeptide sequence. For example, Olig2, HB9, Ngn2, Sox10, or NRx2.2 polypeptide variants include Olig2, HB9, Ngn2, Sox10, or NRx2.2 polypeptides wherein one or more amino acid residues are added or deleted at the N- or C-terminus of the full-length native amino acid sequence. An Olig2, HB9, Ngn2, Sox10, or NRx2.2 polypeptide variant will have at least about 80% amino acid sequence identity, preferably at least about 81% amino acid sequence identity, more preferably at least about 82%, 83%, 84%, 85%, 86%, 87%, 88%, 89%, 90%, 91%, 92%, 93%, 94%, 95%, 96%, 97%, 98% amino acid sequence identity and most preferably at least about 99% amino acid sequence identity with a full-length native sequence Olig2, HB9, Ngn2, Sox10, or NRx2.2 polypeptide sequence. An Olig2, HB9, Ngn2, Sox10, or NRx2.2 polypeptide variant may have a sequence lacking the signal peptide, an extracellular domain of an Olig2, HB9, Ngn2, Sox10, or NRx2.2 polypeptide, with or without the signal peptide, or any other fragment of a full-length Olig2, HB9, Ngn2, Sox10, or NRx2.2 polypeptide sequence. Ordinarily, Olig2, HB9, Ngn2, Sox10, or NRx2.2 variant polypeptides are at least about 10 amino acids in length, often at least about 20 amino acids in length, more often at least about 30, 40, 50, 60, 70, 80, 90, 100, 150, 200, or 300 amino acids in length, or more.

“Percent (%) amino acid sequence identity” is defined as the percentage of amino acid residues that are identical with amino acid residues in a disclosed Olig2, HB9, Ngn2, Sox10, or NRx2.2 polypeptide sequence in a candidate sequence when the two sequences are aligned. To determine % amino acid identity, sequences are aligned and if necessary, gaps are introduced to achieve the maximum % sequence identity; conservative substitutions are not considered as part of the sequence identity. Amino acid sequence alignment procedures to determine percent identity are well known to those of skill in the art. Often publicly available computer software such as BLAST, BLAST2, ALIGN2 or Megalign (DNASTAR) software is used to align peptide sequences. Those skilled in the art can determine appropriate parameters for measuring alignment, including any algorithms needed to achieve maximal alignment over the full length of the sequences being compared.

When amino acid sequences are aligned, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as: % amino acid sequence identity=(X/Y)×100

where

X is the number of amino acid residues scored as identical matches by the sequence alignment program's or algorithm's alignment of A and B

and

Y is the total number of amino acid residues in B.

If the length of amino acid sequence A is not equal to the length of amino acid sequence B, the % amino acid sequence identity of A to B will not equal the % amino acid sequence identity of B to A.

3. Isolated/Purified Polypeptides

An “isolated” or “purified” polypeptide, protein or biologically active fragment is separated and/or recovered from a component of its natural environment. Contaminant components include materials that would typically interfere with diagnostic or therapeutic uses for the polypeptide, and may include enzymes, hormones, and other proteinaceous or non-proteinaceous materials. Preferably, the polypeptide is purified to a sufficient degree to obtain at least 15 residues of N-terminal or internal amino acid sequence. To be substantially isolated, preparations having less than 30% by dry weight of non-Olig2, HB9, Ngn2, Sox10, or NRx2.2 contaminating material (contaminants), more preferably less than 20%, 10% and most preferably less than 5% contaminants. An isolated, recombinantly-produced Olig2, HB9, Ngn2, Sox10, or NRx2.2 or biologically active portion is preferably substantially free of culture medium, that is, culture medium represents less than 20%, more preferably less than about 10%, and most preferably less than about 5% of the volume of the Olig2, HB9, Ngn2, Sox10, or NRx2.2 preparation. Examples of contaminants include cell debris, culture media, and substances used and produced during in vitro synthesis of an Olig2, HB9, Ngn2, Sox10, or NRx2.2.

4. Biologically Active

Biologically active portions of an Olig2, HB9, Ngn2, Sox10, or NRx2.2 include peptides comprising amino acid sequences sufficiently homologous to or derived from the amino acid sequences of an Olig2, HB9, Ngn2, Sox10, or NRx2.2 that include fewer amino acids than a full-length Olig2, HB9, Ngn2, Sox10, or NRx2.2, and exhibit at least one activity of an Olig2, HB9, Ngn2, Sox10, or NRx2.2, such as their activity as a lineage priming agent. Biologically active portions comprise a domain or motif with at least one activity of a native Olig2, HB9, Ngn2, Sox10, or NRx2.2. A biologically active portion of an Olig2, HB9, Ngn2, Sox10, or NRx2.2 can be a polypeptide that is, for example, 10, 25, 50, 100 or more amino acid residues in length. Other biologically active portions, in which other regions of the protein are deleted, can be prepared by recombinant techniques and evaluated for one or more of the functional activities of a native Olig2, HB9, Ngn2, Sox10, or NRx2.2.

5. Determining Homology Between Two or More Sequences

“Olig2, HB9, Ngn2, Sox10, or NRx2.2 variant” means an active Olig2, HB9, Ngn2, Sox10, or NRx2.2 having at least: (1) about 80% amino acid sequence identity with a full-length native sequence Olig2, HB9, Ngn2, Sox10, or NRx2.2 sequence, (2) an Olig2, HB9, Ngn2, Sox10, or NRx2.2 sequence lacking the signal peptide, (3) an extracellular domain of an Olig2, HB9, Ngn2, Sox10, or NRx2.2, with or without the signal peptide, or (4) any other fragment of a full-length Olig2, HB9, Ngn2, Sox10, or NRx2.2 sequence. For example, Olig2, HB9, Ngn2, Sox10, or NRx2.2 variants include an Olig2, HB9, Ngn2, Sox10, or NRx2.2, wherein one or more amino acid residues are added or deleted at the N- or C-terminus of the full-length native amino acid sequence. An Olig2, HB9, Ngn2, Sox10, or NRx2.2 variant will have at least about 80% amino acid sequence identity, preferably at least about 81% amino acid sequence identity, more preferably at least about 82%, 83%, 84%, 85%, 86%, 87%, 88%, 89%, 90%, 91%, 92%, 93%, 94%, 95%, 96%, 97%, 98% amino acid sequence identity and most preferably at least about 99% amino acid sequence identity with a full-length native sequence Olig2, HB9, Ngn2, Sox10, or NRx2.2 sequence. An Olig2, HB9, Ngn2, Sox10, or NRx2.2 variant may have a sequence lacking the signal peptide, an extracellular domain of an Olig2, HB9, Ngn2, Sox10, or NRx2.2, with or without the signal peptide, or any other fragment of a full-length Olig2, HB9, Ngn2, Sox10, or NRx2.2 sequence. Ordinarily, Olig2, HB9, Ngn2, Sox10, or NRx2.2 variant polypeptides are at least about 10 amino acids in length, often at least about 20 amino acids in length, more often at least about 30, 40, 50, 60, 70, 80, 90, 100, 150, 200, or 300 amino acids in length, or more.

### EXAMPLES

### Example 1

### NSFC Cultures

The NSFC cultures were obtained from adult olfactory neuroepithelium, either cadavers or patients via endoscopic biopsy. Procedures for the harvest of these cells from primary cultures have been previously described (see, for example, ADULT HUMAN OLFACTORY PROGENITOR CELLS by Roisen et al., published as WO 03/064601 on 7 Aug. 2003). Once established, NSFC cultures are typically frozen for storage. Frozen stock of early passaged NSFC cell cultures (passages 3 to 6) was thawed rapidly, and 5×10⁵ cells were placed in each flask in minimal essential medium (MEM) with 10% heat-inactivated fetal bovine serum (FBS) (GIBCO, Grand Island, N.Y.), 10 mg/100 ml gentamycin (MEM10) in flasks (25 cm², Corning Incorporated, Corning, N.Y.) in humidified 5% CO₂/95% air (37° C.) for 24 hours. The NSFCs were adapted to the absence of serum via serial dilution of serum every 2 days for 1 week until the cells were finally cultured in DFB27M (DMEM/F12 supplemented with 2% B27) and 10 mg/100 ml gentamycin for 1 week and then used for in vitro analyses between passages 10 and 20. Parallel experiments were preformed on NSFCs with both cultures to determine if patient-specific differences were obtained. Equivalent results were obtained with these two different cultures.

### Example 2

### Lineage Priming of NSFCs with Retinoic Acid, Forskolin, and Sonic Hedgehog

Retinoic acid, Coleus forskohlii forskolin, and a recombinant form of the mouse sonic hedgehog protein produced in E. coli (25-198 of mouse Shh fused to a 6× histidine tag at the carboxy-terminus) were obtained from a commercial supplier (Sigma Chemical Company, St. Louis, Mo.). The NSFCs were plated on glass coverslips in six well plates (3×10⁴ cells/35 mm well) in DFBNM, and treated with various concentrations and combinations of RA, FN and Shh for 7 days {0.5 μM RA (RA0.5), 1 μM RA (RA1), 2 μM RA (RA2), 5 μM FN (FN), 15 nM Shh (Shh), 1 μM RA and 5 μM FN(RA1 FN5), 1 μM RA and 15 nM Shh (RA1 Shh)}. An alternate paradigm provided four days initial treatment with 1 μM RA and 5 μM FN, followed by three days of treatment with 1 μM RA and 15 nM Shh (RFS treatment). After treatment the neurite number, length, and neuritogenic index (neurite numbers×neurite lengths) were determined at 1-7 days in vitro. Cells (500-1,000) were sampled systematically from standardized fields (total magnification 200×) with the aid of an eyepiece reticule under constant magnification with phase contrast optics. Only those primary neurites originating directly from the soma that were longer than the diameter of the cell body were evaluated in a double blind study. Each experiment was performed at least three times with comparable results.

### Example 3

### Construction of Expression Vectors

Full-length mouse Olig2 cDNA, Ngn2, HB9, NRx2.2, and Sox10 were cloned into the pIRES2-enhanced green fluorescent protein (EGFP) expression vector (Clontech, Palo Alto, Calif.) individually (Olig2 and EGFP (O-E), Ngn2 and EGFP (N-E), (HB9 and EPFG (H-E), NRx2.2 and EGFP, and Sox10 and EGFP). For the Olig2 and Ngn2 co-expression vector, Ngn2 cDNA was cloned into pIRES (Clontech) between NheI and EcoRI, and Olig2 cDNA was inserted between XbaI and SalI. For co-expression of Ngn2 and HB9, the Ngn2 cDNA was cloned into the pLHCX expression vector (Clontech), and H-E was used. For co-expression of Olig2 and HB9, the Olig2 cDNA in pCAAGS vectors, and H-E was used. The NRx2.2 gene was isolated by screening the mouse 129Sv cDNA library and cloned into the pIRES2-EGFP expression vector. For the Olig2 and NRx2.2 coexpression vector, Olig2 cDNA was cloned into pIRES (Clontech) between NheI and EcoRI, and NRx2.2 cDNA was inserted between XbaI and SalI. For coexpression of Sox10 and NRx2.2, the chicken Sox10 cDNA and mouse NRx2.2 were sequentially cloned into the pIRES expression vector. All expression vectors were verified by extensive DNA sequencing. The pIRES2-EGFP and pIRES expression vectors served as controls.

### Example 4

### DNA Transfection and Selection

All plasmid constructs were introduced into the NSFCs by liposomal transfection. The cells were plated on glass coverslips in six-well plates (3×10⁴ cells/35-mm well) in either DFB27M or DFBNM without antibiotics one day before transfection. NSFCs were transfected with each plasmid (4 μg/well) for 48 hours according to the manufacturer's protocol (Life Technologies, Rockville, Md.). A further control was provided by lipofectamine alone. Two days after transfection, the cells were fixed or fed with DFB27M supplemented with G418 (50 μg/ml; GIBCO, Grand Island, N.Y.) and/or hygromycin (50 μg/ml; GIBCO) for seven days in vitro. In the case of NSFC's transfected with the vectors that express Olig2, Ngn2, HB9, Olig2 and Ngn2, Ngn2 and HB9, or Olig2 and HB9, following selection, the transfected NSFCs received an RFS treatment.

### Example 5

### MTT (3(4,5-dimethylthiazol-2-O-2,5-diphenyl tetrazolium bromide) assay

The viability of the NSFC after a treatment regimen (7 days treatment with RA, FN, and/or Shh; or 2 days of DNA-transfection of cells and seven days of selection) was measured with a MTT kit (Sigma Chemical Co., St. Louis, Mo.). Cells were plated at a density of 5×10⁴ cells per well in 24-well plates (Falcon). Cells seeded in either DFB27M or DFBNM without treatment served as controls. Mitochondria dehydrogenases in living cells metabolized MTT into formazan crystals, the concentration of which was determined spectrophotometrically at a wavelength of 570 nm. Each experiment was performed at least three times with equivalent results.

### Example 6

### Neurite Formation Induced by Transfected DNAs

NSFCs were plated on glass coverslips in six well plates (3×10⁴ cells/35 mm well) in DFBNM. After transfection and selection combined with the treatment of RFS, the number and length of neurites were determined. Cells (500-1,000) were sampled systematically from standardized fields (total magnification 200×) with the aid of an eyepiece reticule under constant magnification with phase contrast optics. Only those primary neurites originating directly from the soma of the NSFCs that were greater than the diameter of the cell body were evaluated in a double blind study.

### Example 7

### Electron Microscopy

The cultures treated with RA1 FN5Shh were fixed in 3% glutaraldehyde in 0.1 M phosphate buffer at pH 7.4 at 4° C. for 4 h. Following treatment with 1′)/0 osmium tetroxide, dehydration through an ethyl alcohol series and embedment, selected areas were mounted, sectioned, stained with 1% uranyl acetate and lead citrate. The neuritic spines were examined as previously described.

### Example 8

### Immunocytochemistry

The NSFCs (3×10⁴ cells/well) were plated on 22 mm round glass coverslips in 6-well plates (Falcon) and incubated at 37° C. in 5% CO₂/95% air for 24 h; transfected for 2 days; and either immediately fixed or selected for 7 days combine with 4 days treatment with 1 μM RA and 5 μM FN and another 3 days treatment with 1 μM RA and 15 nM Shh (RFS) prior to fixation for immunofluorescence. Cultures were incubated with 4′,6′-diamidino-2-phenylindole dihydrochloride (DAPI) (1:1000, 2 mg/ml, Molecular Probes, Eugene, Oreg.) for 30 min at 37° C. for vital labeling of DNA when nuclear staining was desired. The coverslips were rinsed with cytoskeletal buffer (CB) twice and fixed in 3% paraformaldehyde in CB (10 min), when permeabilization was desired treated with 0.2% Triton X-100 (10 min, Sigma), and incubated (1 h) in 3% bovine serum album (BSA) in Tris-Buffered Saline (TBS). Primary antibodies (See Table 1) were applied overnight (4° C.). After washing (1 h) in TBS three times, the cells were incubated with secondary antibodies: Texas red-conjugated goat anti-rabbit IgG, Texas red-conjugated goat anti-mouse IgG, Cy2-conjugated goat anti-mouse IgG (all diluted 1:100, Cy2, Jackson Immunology Research Laboratories, West Grove, Pa.; Texas red, Molecular Probes, Eugene, Oreg.). Experiments were preformed in triplicate; first and second antibody omission controls were performed with each experiment to ensure the specificity of staining.

TABLE 1
Antibodies and specificity

| Antibodies | Source |
|---|---|
| Nestin, human monoclonal, | Chemicon |
| 1:100 | Temecula, CA |
| Peripherin, polyclonal, | Chemicon |
| 1:100 | |
| B-tubulin III, monoclonal, | Sigma, St. Louis, MO |
| 1:100 | |
| a internexin, polyclonal, | Chemicon |
| 1:100 | |
| Tau, monoclonal, 1:100 | Chemicon |
| NF68, monoclonal, 1:400 | Sigma |
| NF 160, monoclonal, 1:100 | Sigma |
| NF200, monoclonal, 1:100 | Sigma |
| VAChT, polyclonal, 1:200, | Chemicon |
| (WB) 1:1000 | |
| ChAT, monoclonal, 1:200, | Chemicon |
| (WB) 1:1000 | |
| ACh, monoclonal, 1:200 | Chemicon |
| TH, monoclonal, 1:200, | Sigma |

### Example 9

### Western Blot Analysis

Western blot analysis was employed to support the immunofluorescence studies. Proteins from NSFCs cultured in DFBNM without selection, NSFCs transfected with control vectors, as well as NSFCs transfected with the vectors plus each combination of transcriptions factors (Olig2, Ngn2, HB9, Olig2 and Ngn2, Ngn2 and HB9, and Olig2 and HB9), selected and combined with RFS treatment in all groups, were collected in lysis buffer. After 10 min incubation (4° C.), samples were centrifuged at 12,000×g (20 min) and the protein concentration of each supernatant was determined. The protein samples (20 μg/well) were electrophoresed on 10%-14% SDS-polyacrylamide gels along with standardized molecular size marker proteins in an adjacent lane and transferred from gel to nitrocellulose paper. Nonspecific binding was blocked (1 h) with 5% nonfat dry milk in TBST buffer. Blots were incubated (4° C. overnight) following addition of primary antibodies (See Table 1). Blots were washed with TBST buffer four times for 20 min and then three times for 10 min. Washed blots were incubated (1 h) with polyclonal horseradish peroxidase-labeled anti-rabbit IgG (1:1000) as well a monoclonal horseradish peroxidase-labeled anti-mouse IgG (1:1000). Chemiluminescence Western blotting detection (Bio-Rad, Hercules, Calif.) was employed to identify bound antibodies. Densitometry of the protein bands was carried out on a Molecular Dynamics gel scanner (Molecular Dynamics, Sunnyvale, Calif.). Data were analyzed using the Image Quant software programs supplied by the manufacturer.

### Example 10

### BrdU (5-bromo-2′-deoxyuridine) Incorporation

The NSFCs (3×10⁴ cells/well) were plated on 22 mm round glass coverslips in 6-well plates (Falcon, Franklin Lakes, N.J.) and incubated at 37° C. in 5% CO₂/95% air. To examine mitotic activity of NSFCs after transfection and selection combined with RA1 FN5Shh treatment, 5-bromo-2′ deoxyuridine (BrdU, 10 μM, Sigma) was added to the cells for 24 h before fixation. The cells were rinsed with cytoskeletal buffer twice and fixed in 3% paraformaldehyde in CB (10 min), when permeabilization was desired treated with 0.2% Triton X-100 (10 min, Sigma), and incubated in 0.6% H₂O₂ in Tris-Buffered Saline (TBS) for 30 min. Cells were incubated in 2 N HCl for 30 min at 37° C. Acid was removed by washing with TBS twice and neutralized with 0.1 M sodium borate (Sigma) for 10 min. Cells were incubated (1 h) in 3% bovine serum album (BSA) in TBS. Primary antibody anti-BrdU was applied overnight (4° C.). After washing (1 h) in TBS three times, the cells were incubated with secondary antibodies: Cy2-conjugated goat anti-mouse IgG, or Texas red-conjugated goat anti-mouse IgG (1:100, Cy2, Jackson Immunology Research Laboratories). Experiments were preformed in triplicate; first and second antibody omission controls were performed with each experiment to ensure the specificity of staining.

### Example 11

### Co-Culture

Chicken skeletal pectoral muscles were removed from 12 day embryos and dissociated with 0.25% trypsin at 37° C. for 15 min and plated in 6-well plates (5×10⁵ cells/well) with DF+10% FBS. From two days in vitro, the cells were treated with cytosine β-D-arabinofuranoside (5 μM; Sigma) to eliminate dividing cells and therefore substantially reduce the presence of fibroblasts. After seven days in vitro, cells were trypsinized (0.05% trypsin in Ca²⁺/Mg²⁺-free HBSS), and plated on glass coverslips in 6-well plates with DF+2% FBS for three days. Seven day RA1 FN5Shh-treated NSFCs were co-cultured with the muscle cells for another seven days in DFBNM with RA1 FN5Shh. For the NSFCs that were transfected with Ngn2 and HB9 or Olig2 and HB9, the cells were treated with 1 μM RA and 5 μM FN, and selection was carried out for four days (RF). These NSFCs were then co-cultured with the established muscle straps in DFBNM supplemented with 1 μM RA and 15 nM Shh for an additional three days in DFBNM (RS).

### Example 12

### Statistics

Statistical analysis (Graph pad Prism) was carried out using ANOVA (significance level p<0.05). Cells (500-1,000) were sampled systematically from standardized fields (total magnification 200×) of cells stained for each marker. The mean and standard deviation of triplicate samples repeated a minimum of three times was determined for the NSFCs cultures. There were no detectable differences between the cell cultures.

### Example 13

### Immunomagnetic Separation of Cells

Superparamagnetic polystyrene beads, conjugated with a human anti-mouse IgG, which binds all mouse IgG antibodies (Cellection™ Pan Mouse IgG Kit, Dynal Biotech, Oslo, Norway) were prepared according to the manufacturer's instructions with few modifications. Briefly the beads were obtained at a concentration of (0.5-1×10⁷/per vial); they were suspended in phosphate buffered saline (PB S/0. 1% bovine serum albumin, BSA) and washed 3 times. Primary antibody (Ab), mouse monoclonal Trk-pan (0.2-1 mg, Santa Cruz Biotech, Santa Cruz, Calif.) was added to the bead suspension in PBS with 0.1 BSA. This bead/Ab suspension was rotated for 30 min at room temperature using a sample mixer (Dynal, Oslo, Norway). The tube was placed in a Dynal magnetic particle concentrator (MPC, Dynal) to condense the beads for 1 min and the beads were washed 3 times with PBS with 0.1% BSA.

The NSFCs (100×10⁴/ml buffer) were cooled to 4° C. and the beads were added to the cell suspension to establish a ratio of 5-10 beads per cell. The beads and cells were mixed and incubated for 30 min at 4° C. with gentle tilting and rotation in a Dynal sample mixer. The cells that were positive for Trk-pan attached to the beads and the tube was placed in the MPC to separate positive cells from the heterogeneous population. The supernatant suspension (containing the negative cells) was aspirated, placed in flasks and incubated at 37° C. with 95% air and 5% CO₂ for future use. The rosetted positive cells were resuspended in RPMI medium 1640 (GIBCO) with 1% fetal calf serum at 37° C. The releasing buffer (4 μl DNAse, provided with the bead kit) was added to the rosetted cells to separate the beads from the cells and the solution was incubated for 15 min at 25° C. with gentle tilting and rotation. The solution was flushed vigorously through a Pasteur pipette several times and the tube was placed in the MPC for 1 minute; the supernatant containing the released cells was added to a tube containing RPMI with 10% FCS. The positive cells were incubated at 37° C. with 95% air and 5% CO₂.

### Example 14

### Autologous Transplantation (Prophetic Example)

A patient with multiple sclerosis would have a nasal endoscopic biopsy performed following procedures developed for sampling, isolation and expansion of NSFCs. The NSFCs will undergo high efficiency lineage priming in the lab directed toward oligodendrocytic development. Prior to and after lineage priming samples of the patient's cells will be preserved under liquid nitrogen for future treatment. Aliquots of the lineage primed cells will be prepared and sent to the neurosurgeon for autologous transplantation into selected regions of the brain and spinal cord as determined by MRI or other imaging techniques. The dosage will be approximately 80,000 cells/μl with a range of 30-40 μl/site.

A patient with motor neuronal degenerative diseases such as Parkinson's disease or ALS would undergo similar treatment with neuronal lineage primed NSFCs. For the patient with Parkinson's disease, following nasal endoscopic biopsy, isolated NSFCs will be lineage primed towards dopaminergic neurons (Zhang et al., 2006). Prior to and after lineage priming samples of the patient's cells will be preserved under liquid nitrogen for future treatment. Aliquots of lineage primed cells will be prepared and sent to the neurosurgeon for autologous transplantation into the substantia nigra bilaterally with 30-40 μl of solution/site (80,000 cells/μl). Similarly, a patient with ALS would undergo the biopsy and NSFCs isolation procedures as described previously. The patient's cells would be lineage primed toward motor neuronal development as reported by our group (Zhang et al. 2005). Prior to and after lineage priming samples of the patient's cells will be preserved under liquid nitrogen for future treatment. Aliquots of the lineage primed cells will be prepared and sent to the neurosurgeon for transplantation into the ventral horn (motor nucleus) of the patient's spinal cord in predetermined patient specific sites.

1. A method of transplantation, comprising:
lineage priming human progenitor cells, to form lineage primed cells; and
transplanting the lineage primed cells into a patient;
wherein the lineage primed cells are selected from the group consisting of oligodendrocytic lineage primed cells, dopaminergic lineage primed cells, and motoneuronal lineage primed cells, and
lineage priming has an efficiency of at least 1%.

2. The method of claim 1, wherein the human progenitor cells are adult human progenitor cells.

3. The method of claim 2, wherein the human progenitor cells are neurosphere forming cells.

4. The method of claim 3, wherein the lineage priming has an efficiency of at least 5%.

5. The method of claim 3, wherein the lineage priming has an efficiency of at least 10%.

6. The method of claim 3, wherein the lineage primed cells are oligodendrocytic lineage primed cells.

7. The method of claim 3, wherein the lineage primed cells are dopaminergic lineage primed cells.

8. The method of claim 3, wherein the lineage primed cells are motoneuronal lineage primed cells.

9. The method of claim 3, further comprising culturing human tissue, to form the neurosphere forming cells; wherein the human tissue comprises olfactory neuroepithelium.

10. The method of claim 9, wherein the culturing of the human tissue comprises:
an initial culturing of the human tissue;
inducing the human tissue to form neurosphere forming cells; and
subculturing the neurosphere forming cells.

11. The method of claim 9, further comprising collecting the human tissue from a living donor.

12. The method of claim 11, wherein the living donor is the patient.

13. The method of claim 12, wherein the patient has a neurological disorder.

14. The method of claim 13, wherein the lineage primed cells are oligodendrocytic lineage primed cells, and neurological disorder is multiple sclerosis.

15. The method of claim 13, wherein the lineage primed cells are dopaminergic lineage primed cells, and the neurological disorder is Parkinson's disease.

16-19. (canceled)

20. A method for producing lineage primed cells, comprising:
lineage priming human progenitor cells, to form lineage primed cells;
wherein the lineage priming has an efficiency of at least 1%, and
the lineage primed cells are dopaminergic lineage primed cells or motoneuronal lineage primed cells.

21-36. (canceled)

37. A lineage priming composition, comprising retinoic acid, and at least one of sonic hedgehog and forskolin.

38-40. (canceled)

41. A lineage priming composition, comprising HB9, and at least one of Olig2 and Ngn2, wherein the composition causes lineage priming of neurosphere forming cell with an efficiency of at least 1%.

42-43. (canceled)

44. A cell culture, comprising neurosphere forming cells and at least 1% dopaminergic lineage primed cells.

45-46. (canceled)

47. A cell culture, comprising neurosphere forming cells and at least 1% motoneuronal lineage primed cells.

48-49. (canceled)
